(19) United States
(12) Patent Application Publication
(10) Pub. No.: US 2002/0006438 A1
(43) Pub. Date: Jan. 17, 2002

(54) SUSTAINED RELEASE HYDROMORPHONE FORMULATIONS EXHIBITING BIMODAL CHARACTERISTICS

(76) Inventors: BENJAMIN OSHLACK, NEW YORK, NY (US); MARK CHASIN, MANALAPAN, NJ (US); HUA-PIN HUANG, Englewoods Cliff, NJ (US)

Correspondence Address: CLIFFORD M DAVIDSON, DAVIDSON DAVIDSON & KAPPEL, 1140 AVENUE OF THE AMERICAS, 15TH FLOOR, NEW YORK, NY, 10036

(21) Appl. No.: 09/161,178

(22) Filed: Sep. 25, 1998

(51) Int. Cl. A61K 31/44; A01N 43/42; A61K 47/00; A61K 9/66; A61K 9/14; A61K 9/62; A61K 9/60; A61K 9/56; A61K 9/52; A61K 9/64; A61K 9/22; A61K 9/26; A61K 9/24

(52) U.S. Cl. 424/457; 424/456; 424/455; 424/459; 424/461; 424/468; 424/469; 424/470; 424/471; 424/472; 514/282; 424/439

(57) ABSTRACT

The invention is related to a solid sustained release once-a-day oral dosage form comprising hydromorphone or a pharmaceutically acceptable salt thereof together with a sustained release carrier, the dosage providing a relatively rapid rise in plasma concentration to an initial early peak concentration, followed by a second broader peak with plateau plasma concentrations.

### BACKGROUND OF THE INVENTION

It is known in the pharmaceutical art to prepare compositions which provide for sustained release of pharmacologically active substances contained in the compositions after oral administration to humans and animals. Sustained release formulations known in the art include specially coated pellets, coated tablets and capsules wherein the slow release of the active medicament is brought about through selective breakdown of the coating of the preparation or through compounding with a special matrix to affect the release of a drug. Some sustained release formulations provide for related sequential release of a single dose of an active compound at predetermined periods after administration.

It is the intent of all sustained release formulations to provide a longer period of pharmacologic action after administration than is ordinarily obtained after administration of immediate-release dosage forms. Sustained release compositions may be used to delay absorption of a medicament until it has reached certain portions of the alimentary tract, and maintain a desired concentration of said medicament in the blood stream for a longer duration than would occur if conventional rapid release dosage forms are administered. Such longer periods of response provide for many therapeutic benefits that are not achieved with corresponding short acting, immediate release preparations. Thus, therapy may be continued without interrupting the sleep of the patient, which is of special importance, for example, when treating a patient for moderate to severe pain (e.g., a post-surgery patient, a cancer patient, etc.), or for those patients who experience migraine headaches on awakening, as well as for the debilitated patient for whom sleep is essential. A further general advantage of longer acting drug preparations is improved patient compliance resulting from the avoidance of missed doses through patient forgetfulness.

Unless conventional rapid acting drug therapy is carefully administered at frequent intervals to maintain effective steady state blood levels of the drug, peaks and valleys in the blood level of the active drug occurs because of the rapid absorption, systemic excretion of the compound and through metabolic inactivation, thereby producing special problems in maintenance therapy of the patient.

Due to the difficulties presented by the pharmacotherapy of pain, particularly chronic pain, opioid analgesics are ideal drugs to be administered as controlled release formulations. Certain sustained-release opioid analgesic formulations are commercially available. For example, morphine, which is considered to be the prototypic opioid analgesic, has been formulated into 12 hour controlled-release formulations (i.e., MS Contin® tablets, commercially available from Purdue Frederick Company). More recently, oxycodone has been formulated into 12 hour controlled-release formulation (i.e., OxyContin® tablets, commercially available from Purdue Pharma). Various techniques have been used to prepare controlled release dosage forms. Specially coated pellets, tablets and capsules wherein the slow release of the active medicament is brought about through selective breakdown of the coating of the preparation or through compounding with a special matrix to affect the release of a drug are known in the art. Certain controlled release formulations provide for related sequential release of a single dose of an active compound at predetermined periods after administration.

A further example of controlled release opioid formulations include, for example, those disclosed in U.S. Pat. Nos. 4,990,341 and 4,844,909 (Goldie, et al.), both assigned to the assignee of the present invention and incorporated herein by reference, describe hydromorphone compositions wherein the dissolution rate in-vitro of the dosage form, when measured by the USP Paddle or Basket Method at 100 rpm in 900 ml aqueous buffer (pH between 1.6 and 7.2) at 37° C., is between 12.5 and 42.5% (by wt) hydromorphone released after 1 hour, between 25 and 55% (by wt) released after 2 hours, between 45 and 75% (by wt) released after 4 hours and between 55 and 85% (by wt) released after 6 hours, the in-vitro release rate being independent of pH between pH 1.6 and 7.2 and chosen such that the peak plasma level of hydromorphone obtained in-vivo occurs between 2 and 4 hours after administration of the dosage form. At least 12 hours of pain relief is obtained with these hydromorphone formulations.

It has been a further goal in the art to develop drug formulations which provide a duration of effect longer than 12 hours, so that, for example, the drug may be administered to the patient only once a day. Advantageously, once daily dosing formulations provide both increased convenience and compliance, as have been documented for numerous medication formulations when the requirement for less frequent dosing is provided.

Despite the foregoing advances and the various techniques for preparing controlled release formulations available in the pharmaceutical art, there is a need in the art for an orally administrable opioid formulation which would provide an extended duration of effect. Experience with strong analgesics such as MS Contin^{®} Tablets (controlled-release morphine sulfate) has shown that twice daily (q12 h) oral administration leads to improved compliance as compared to immediate-release analgesics dosed every 4 hours (q4 h). Evidence suggests that once-daily administration of many drugs is associated with even greater compliance.

### SUMMARY OF THE INVENTION

It is an object of the present invention to provide bioavailable hydromorphone analgesic formulations which substantially improve the efficiency and quality of pain management.

It is a further object of the present invention to provide an orally administered pharmaceutical dosage form of a hydromorphone that is suitable for once-a-day administration.

It is a further object of the present invention to provide oral dosage forms of hydromorphone or a pharmaceutically acceptable salt thereof which are administrable on a once-a-day basis and which provide effective treatment of pain in mammals, particularly humans.

The above objects and others are attained by virtue of the present invention, which provides a solid sustained release once-a-day oral dosage form comprising hydromorphone or a pharmaceutically acceptable salt thereof together with a sustained release carrier, the dosage providing a relatively rapid rise in plasma concentration to an initial early peak concentration, followed by a second broader peak with plateau plasma concentrations. In certain preferred embodiments, the second broader peak is maintained to 24 hours. The hydromorphone sustained release dosage forms of the invention are preferably bioavailable and preferably provide effective treatment of pain for about 24 hours or more after administration to a mammal, e.g., human.

In preferred embodiments, the sustained release hydromorphone formulations of the invention provide a first time to peak plasma concentration (Tmax #1) of the hydromorphone in about 0.3 to about 4 hours after oral administration of the dosage form to the patient. In certain preferred embodiments, the first time to peak plasma concentration occurs from about 1 to about 3 hours after oral administration. In preferred embodiments, the maximum plasma concentration of hydromorphone at the first Tmax (Cmax #1) is from about 1 to about 3 ng/ml, per administration of a 12 mg dosage of hydromorphone hydrochloride in an oral sustained release dosage form in accordance with the invention. Because the dosage of hydromorphone in the sustained release oral formulations of the invention is dose-proportional, one can easily determine the maximum plasma concentrations for Tmax #1 for different dosages of hydromorphone over a 24 hour period (this holds true for the Cmax #2 as well).

In further preferred embodiments of the invention, the sustained release hydromorphone iformulations provide a second peak plasma concentration (Cmax #2) which occurs in about 10 to about 19 hours after oral administration of the dosage form to the patient (Tmax #2). In certain preferred embodiments, the second peak plasma concentration (Cmax #2) occurs in about 12.5 to about 16 hours after oral administration of the dosage form to the patient (Tmax #2). In preferred embodiments, the maximum plasma concentration of hydromorphone at Cmax #2 is from about 1.0 to about 3.6 ng/ml, per 12 mg hydromorphone administered over the 24 hour period.

In certain preferred embodiments, the W50 of Cmax #1 (defined for purposes of the present invention as the width of the plasma concentration curve at 50% of the height of the first Cmax (Cmax #1), based on a trough taken either as the Cmin between Cmax #1 and Cmax #2 or the plasma concentration at 24 hours after administration of the dose of hydromorphone) is from about 1.5 to about 4.5 hours, preferably from about 2.5 to about 3.5 hours.

In certain preferred embodiments, the W50 of Cmax #2 (defined for purposes of the present invention as the width of the plasma concentration curve at 50% of the height of the second Cmax (Cmax #2), based on a the trough taken either as the Cmin between Cmax #1 and Cmax #2 or the plasma concentration at 24 hours after administration of the dose of hydromorphone) is from about 4.5 to about 9 hours, preferably from about 5.5 to about 7 hours.

In certain preferred embodiments, the sustained release hydromorphone formulations of the invention provide a maximum hydromorphone plasma concentration which is less than twice the plasma level of hydromorphone at about 24 hours after administration of the dosage form.

In certain further preferred embodiments, the sustained release hydromorphone formulations of the invention provide a maximum hydromorphone plasma concentration which is less than twice the plasma level of hydromorphone at the Cmin which occurs between Cmax #1 and Cmax #2.

In certain further preferred emodiments, the sustained release hydromorphone formulations of the invention may be characterized by other pharmacokinetic values which are set forth in the data provided in the appended examples, which data can be readily gleaned by one of ordinary skill in the art reviewing the appended Tables and Figures. Such pharmacokinetic values may be derived in part based on parameters such asCss,max (ng/mL); Css,min (ng/mL); Ct,min (ng/mL); tss,max (hr); fluctuation (%)(expressed as the difference between Css,max and Css,min expressed as a percentage of Css,min); Tss (days), and any combination thereof.

In certain preferred embodiments, the sustained release hydromorphone formulations of the invention provide an in-vitro dissolution of from about 5% to about 25% hydromorphone released after 1 hour; from about 40% to about 75% hydromorphone released after 8 hours; and not less than about 80% hydromorphone released after 18 hours. In additional preferred embodiments, the sustained release hydromorphone formulations of the invention provide an in-vitro dissolution of from about 10% to about 30% hydromorphone released after 2 hours; from about 40% to about 70% hydromorphone released after 8 hours; and at least about 80% hydromorphone released after 22 hours. The in-vitro dissolution method may be one of those set forth in the appended examples. Alternatively, the in-vitro dissolution method may be, e.g., the USP Paddle or Basket Method at 100 rpm in 900 ml aqueous buffer (pH between 1.6 and 7.2) at 37° C.

In certain preferred embodiments, the once-a-day sustained release oral dosage forms of hydromorphone of the present invention are prepared using ingredients and methods set forth in detail herein with respect to the discussion concerning melt extrusion techniques.

The once-a-day sustained release oral dosage forms of hydromorphone of the present invention may be pH independent, e.g., the in-vitro release rate being independent of pH between pH 1.6 and 7.2. Alternatively, the once-a-day sustained release oral dosage forms of hydromorphone of the present invention may be pH dependent.

The Tmax #1 and Tmax #2 values, the Cmax #1 and Cmax #2 values, and other pharmacokinetic parameters used to describe the present invention, are applicable to human patients, whether based on the administration of the dosage form to an individual patient, or when viewed as mean values over a population of human patients.

The term “hydromorphone” is defined for purposes of the present invention as any pharmaceutically acceptable form of the drug, preferably in the form of the hydrochloride salt, but alternatively in the form of molar equivalent amounts of other hydromorphone salts or of the hydromorphone base.

The term “sustained release” is defined for purposes of the present invention as the release of the drug (opioid analgesic) from the transdermal formulation at such a rate that blood (e.g., plasma) concentrations (levels) are maintained within the therapeutic range (above the minimum effective analgesic concentration or “MEAC”) but below toxic levels over a period of time of about 12 hours or longer.

The term “steady state” means that the blood plasma concentration curve for a given drug has been substantially repeated from dose to dose.

The term “minimum effective analgesic concentration” is defined for purposes of this invention as the minimum effective therapeutic blood plasma level of the drug at which at least some pain relief is achieved in a given patient. It will be well understood by those skilled in the medical art that pain measurement is highly subjective and great individual variations may occur among patients.

The term “pH-dependent” for purposes of the present invention is defined as having characteristics (e.g. dissolution) which vary according to environmental pH (e.g., due to changes in the in-vitro dissolution media, or due to passage of the dosage form through the gastrointestinal tract..

The term “pH-independent” for purposes of the present invention is defined as having characteristics (e.g., dissolution) which are substantially unaffected by pH, in that a difference, at any given time, between an amount of opioid released at one pH and an amount released at any other pH, when measured in-vitro using the USP Paddle Method of U.S. Pharmacopeia XXII (1990) at 100 rpm in 900 ml aqueous buffer, is no greater than 10%.

### BRIEF DESCRIPTION OF THE DRAWINGS

The following drawings are illustrative of embodiments of the invention and are not meant to limit the scope of the invention as encompassed by the claims.

FIG. 1 is a graphical representation of the dissolution (mean percent dissolved over time) for Examples 1 and 2.

FIG. 2 is a graphical representation of the concentration of Example 3 (fed and fasted) over time, versus Dilaudid.

FIG. 3 is a graphical representation of the concentration of Example 4 (fed and fasted) over time, versus Dilaudid.

FIG. 4 is a graphical representation of the plasma concentration over time for Example 7.

FIG. 5 is a graphical representation of the plasma concentration over time for Example 8.

FIG. 6 provides the mean plasma concentration over time (up to 30 hours) for HHCR 12 mg, HHCR 24 mg, and Dilaudid.

FIG. 7 provides the mean plasma concentration-time course data for Example 12.

FIG. 8 provides the mean trough values (ng/ml) over time for HHIR and HHCR.

FIG. 9 provides the mean subject drug effect (VAS, mm) over time for HHIR and HHCR.

FIG. 10 provides a graphical representation of the mean plasma hydromorphone concentration (ng/ml) and mean subject drug effect (VAS, mm) over time for HHCR.

FIG. 11 provides a graphical representation of the mean plasma hydromorphone concentration (ng/ml) and mean subject drug effect (VAS, mm) over time for HHIR.

### DETAILED DESCRIPTION

The sustained release formulations of the present invention include hydromorphone as the therapeutically active opioid in an amount from about 2 mg to about 64 mg or more hydromorphone hydrochloride. Alternatively, the dosage form may contain molar equivalent amounts of other hydromorphone salts or of the hydromorphone base.

The sustained release dosage forms of the present invention generally achieve and maintain therapeutic levels substantially without significant increases in the intensity and/or degree of concurrent side effects, such as nausea, vomiting or drowsiness, which are often associated with high blood levels of opioid analgesics. There is also evidence to suggest that the use of the present dosage forms leads to a reduced risk of drug addiction.

The sustained release hydromorphone formulations of the present invention are preferably bioavailable. It is generally recognized that the mere presence of an active substance in the gastrointestinal fluids does not, by itself, insure bioavailability. In order to be absorbed, the active drug substance must be in solution. The time required for a given proportion of an active substance from a unit dosage form is determined as the proportion of the amount of active drug substance released from a unit dosage form over a specified time base by a test method conducted under standardized conditions. The physiologic fluids of the gastrointestinal tract are the media for determining dissolution time. The present state of the art recognizes many satisfactory test procedures to measure dissolution time for pharmaceutical compositions, and these test procedures are described in official compendia world-wide.

Although there are many diverse factors which influence the dissolution of drug substance from its carrier, the dissolution time determined for a pharmacologically active substance from the specific composition is relatively constant and reproducible. Among the different factors affecting the dissolution time are the surface area of the drug substance presented to the dissolution solvent medium, the pH of the solution, the solubility of the substance in the specific solvent medium, and the driving forces of the saturation concentration of dissolved materials in the solvent medium. Thus, the dissolution concentration of an active drug substance is dynamically modified in its steady state as components are removed from the dissolution medium through absorption across the tissue site. Under physiologic conditions, the saturation level of the dissolved materials is replenished from the dosage form reserve to maintain a relatively uniform and constant dissolution concentration in the solvent medium providing for a steady state absorption.

The transport across a tissue absorption site of the gastrointestinal tract is influenced by the Donnan osmotic equilibrium forces on both sides of the membrane since the direction of the driving force is the difference between the concentrations of active substance on either side of the membrane, i.e., the amount dissolved in the gastrointestinal fluids and the amount present in the blood. Since the blood levels are constantly being modified by dilution, circulatory changes, tissue storage, metabolic conversion and systemic excretion, the flow of active materials is directed from the gastrointestinal tract into the blood stream.

Notwithstanding the diverse factors influencing both dissolution and absorption of a drug substance, a strong correlation has been established between the in-vitro dissolution time determined for a dosage form and (in-vivo) bioavailability. The dissolution time and the bioavailability determined for a composition are two of the most significant fundamental characteristics for consideration when evaluating sustained-release compositions. Surprisingly however, the dissolution profiles of the once-a-day sustained release oral hydromorphone formulations of the invention are not predictive of the bimodal plasma concentration curve (meaning the occurrence of a Tmax #1 and Tmax #2 as described herein).

Sustained release dosage forms having the desired inventive characteristics can be formulated as a pharmaceutically acceptable tablet, caplet, or multiparticulate formulation known to those skilled in the art. The sustained release dosage form may optionally include a sustained released carrier which is incorporated into a matrix along with the hydromorphone, or which is applied as a sustained release coating.

The sustained release dosage form may include a portion of the hydromorphone in sustained release form and remaining portion of the hydromorphone in immediate release form. For example, the sustained release dosage form may have a relatively larger portion of the hydromorphone in sustained release form and a smaller portion of the hydromorphone incorporated into the dosage form in immediate release form.

An oral dosage form according to the invention may be provided as, for example, granules, spheroids, beads, pellets (hereinafter collectively referred to as “multiparticulates”) or a tablet. An amount of the multiparticulates effective to provide the desired dose of hydromorphone over time may be placed in a capsule or may be incorporated in any other suitable oral solid form.

### Sustained Release Coatings

In certain preferred embodiments, the hydromorphone is incorporated into or onto a substrate and a sustained release coating is applied thereto. For example, the hydromorphone may be contained within or on a substrate as follows: (i) incorporated into matrix spheroids (e.g., together with a pharmaceutically acceptable spheronizing agent such as microcrystalline cellulose), (ii) coated onto inert pharmaceutically acceptable beads (e.g., nonpareil beads); (iii) incorporated into a normal release tablet core; or (iv) incorporated into a tablet core which comprises a matrix including a sustained release carrier material. Thereafter, a sustained release coating is applied onto substrates such as those mentioned in (i)-(iv) above. The dosage forms of the present invention may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the opioid in desired areas of the gastro-intestinal (GI) tract, e.g., the stomach or small intestine, such that an absorption profile is provided which is capable of providing at least about twelve hour and preferably up to twenty-four hour analgesia to a patient. When a pH-independent coating is desired, the coating is designed to achieve optimal release regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings to obtain formulations may also impart a repeat-action effect whereby unprotected drug is coated over the enteric coat and is released in the stomach, while the remainder, being protected by the enteric coating, is released further down the gastrointestinal tract. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

In certain preferred embodiments, the substrate (e.g., tablet core bead, matrix particle) comprising the hydromorphone is coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile. Such formulations are described, e.g., in detail in U.S. Pat. Nos. 5,273,760 and 5,286,493, assigned to the Assignee of the present invention and hereby incorporated by reference. The particles are preferably film coated with a material that permits release of the hydromorphone so as to achieve, in combination with the other stated properties, a desired in-vitro release rate and in-vivo plasma levels. The sustained release coating formulations of the present invention should be capable of producing a strong, continuous film that is smooth and elegant, capable of supporting pigments and other coating additives, non-toxic, inert, and tack-free.

Other examples of sustained release formulations and coatings which may be used in accordance with the present invention include Assignee's U.S. Pat. Nos. 5,324,351; 5,356,467, and 5,472,712, hereby incorporated by reference in their entirety.

Alkylcellulose Polymers

Cellulosic materials and polymers, including alkylcelluloses, provide hydrophobic materials well suited for coating the beads according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially-available aqueous dispersion of ethylcellulose is Aquacoat® (FMC Corp., Philadelphia, Penna., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Penna, U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Acrylic Polymers

The hydrophobic material comprising the controlled release coating may comprise a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Röhm Tech, Inc. There are several different types of Eudragit®. For example, Eudragit® E is an example of a methacrylic acid copolymer which swells and dissolves in acidic media. Eudragit® L is a methacrylic acid copolymer which does not swell at about pH<5.7 and is soluble at about pH >6. Eudragit® S does not swell at about pH <6.5 and is soluble at about pH>7. Eudragit® RL and Eudragit® RS are water swellable, and the amount of water absorbed by these polymers is pH-dependent, however, dosage forms coated with Eudragit® RL and RS are pH-independent.

In certain preferred embodiments, the acrylic coating comprises a mixture of two acrylic resin lacquers commercially available from Rohm Pharma under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L.

Plasticizers

In embodiments of the present invention where the coating comprises an aqueous dispersion of a hydrophobic material such as an alkylcellulose or an acrylic polymer, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

When the aqueous dispersion of hydrophobic material is used to coat a substrate including the hydromorphone, for example, inert pharmaceutical beads such as nu pariel 18/20 beads, a plurality of the resultant stabilized solid controlled release beads may thereafter be placed in a gelatin capsule in an amount sufficient to provide an effective controlled release dose when ingested and contacted by an environmental fluid, e.g., gastric fluid or dissolution media. Alternatively, the substrate may be a tablet core coated with the sustained release coating, and optionally a further film-forming agent or colorant, such as Opadry®.

In formulations where an aqueous dispersion of an hydrophobic polymer such as an alkylcellulose is applied to the substrate, it is preferred that the coated substrate is cured at a temperature above the glass transition temperature of the plasticized polymer and at a relative humidity above ambient conditions, until an endpoint is reached at which the coated formulation attains a dissolution profile which is substantially unaffected by exposure to storage conditions, e.g., of elevated temperature and/or humidity. Generally, in such formulations the curing time is about 24 hours or more, and the curing conditions may be, for example, about 60° C. and 85% relative humidity. Detailed information concerning the stabilization of such formulations is set forth in U.S. Pat. Nos. 5,273,760; 5,681,585; and 5,472,712; all of which are hereby incorporated by reference in their entireties.

In formulations where an aqueous dispersion of an acrylic polymer is applied to the substrate, it is preferred that the coated substrate is cured at a temperature above the glass transition temperature of the plasticized polymer until an endpoint is reached at which the coated formulation attains a dissolution profile which is substantially unaffected by exposure to storage conditions, e.g., of elevated temperature and/or humidity. Generally, the curing time is about 24 hours or more, and the curing temperature may be, for example, about 45° C. Detailed information concerning the stabilization of such formulations is set forth in U.S. Pat. Nos. 5,286,493; 5,580,578; and 5,639,476; all of which are hereby incorporated by reference in their entireties.

The sustained release profile of the coated formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the retardant coating.

Spheroids or beads coated with a therapeutically active agent are prepared, e.g., by dissolving the therapeutically active agent in water and then spraying the solution onto a substrate, for example, nu pariel 18/20 beads, using a Wuster insert. Optionally, additional ingredients are also added prior to coating the beads in order to assist the binding of the opioid to the beads, and/or to color the solution, etc. For example, a product which includes hydroxypropylmethylcellulose, etc. with or without colorant (e.g., Opadry®, commercially available from Colorcon, Inc.) may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic controlled release coating. An example of a suitable barrier agent is one which comprises hydroxypropylmethylcellulose. However, any film-former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethyl-cellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color be added to Aquacoat® via the use of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

The plasticized aqueous dispersion of hydrophobic material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined sustained release of the therapeutically active agent (i.e., hydromorphone) when the coated substrate is exposed to aqueous solutions, e.g. gastric fluid, is preferably applied, taking into account the physical characteristics of the therapeutically active agent, the manner of incorporation of the plasticizer, etc. After coating with the hydrophobic material, a further overcoat of a film-former, such as Opadry®, is optionally applied to the beads. This overcoat is provided, if at all, in order to substantially reduce agglomeration of the beads.

The release of the hydromorphone from the sustained release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion-promoting agents such as starch and gums.

The sustained release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer.

In certain preferred embodiments, the release-modifying agent is selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864 (all of which are hereby incorporated by reference). The passageway can have any shape such as round, triangular, square, elliptical, irregular, etc.

### Sustained Release Matrices

In certain preferred embodiments of the present invention, the sustained release formulation comprises a matrix including the hydromorphone and a sustained release carrier (which may comprise one or more hydrophobic materials, such as an alkylcellulose and/or an acrylic polymer as previously defined herein). The materials suitable for inclusion in a sustained release matrix will depend on the method used to form the matrix.

Suitable materials for inclusion in the sustained release matrices of the invention, in addition to the hydromorphone, include:

(A) hydrophilic and/or hydrophobic materials, such as gums; alkylcelluloses; cellulose ethers, including hydroxyalkylcelluloses and carboxyalkylcelluloses; acrylic resins, including all of the acrylic polymers and copolymers discussed above, and protein derived materials. This list is not meant to be exclusive, and any pharmaceutically acceptable hydrophobic material or hydrophilic material which is capable of imparting the desired sustained release profile of the hydromorphone is meant to be included herein. The dosage form may comprise, e.g., from about 1% to about 80% by weight of such material.

In certain preferred embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the hydrophobic material is selected from materials such as hydroxyalkylcelluloses such as hydroxypropyl-methylcellulose and mixtures of the foregoing. In yet other embodiments, the hydrophobic material is an alkylcellulose.

(B) digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and natural or synthetic waxes, polyhydric alcohols, including polyalkylene glycols. The oral dosage form may contain up to 60% (by weight) of such material. In certain embodiments, a combination of two or more hydrocarbon materials are included in the matrix formulations. If an additional hydrocarbon material is included, it is preferably selected from natural and synthetic waxes, fatty acids, fatty alcohols, and mixtures of the same.

Preferred hydrocarbons are water-insoluble with more or less pronounced hydrophilic and/or hydrophobic trends, and have a melting point from about 30° to about 200° C., preferably from about 45° to about 90° C.

For purposes of the present invention, a wax-like substance is defined as any material which is normally solid at room temperature and has a melting point of from about 30° to about 100° C. Suitable waxes include, for example, beeswax, glycowax, castor wax and carnauba wax.

The aliphatic alcohol may be, for example, lauryl alcohol, myristyl alcohol or stearyl, cetyl and/or cetostearyl alcohol. The amount of aliphatic alcohol, if included in the present oral dosage form, will be determined, as above, by the precise rate of hydromorphone release required. In certain embodiments, the oral dosage form contains between 20% and 50% (by wt) aliphatic alcohol. When at least one polyalkylene glycol is present in the oral dosage form, then the combined weight of the at least one aliphatic alcohol and the at least one polyalkylene glycol preferably constitutes between 20% and 50% (by wt) of the total dosage.

In one embodiment, the ratio of, e.g., the at least one hydroxyalkyl cellulose or acrylic resin to the at least one aliphatic alcohol/ polyalkylene glycol determines, to a considerable extent, the release rate of the opioid from the formulation.

Suitable polyalkylene glycols include, for example, polypropylene glycol or polyethylene glycol. The number average molecular weight of the at least one polyalkylene glycol is preferred between 1,000 and 15,000 especially between 1,500 and 12,000.

In addition to the above ingredients, a controlled release matrix may also contain suitable quantities of other materials, e.g. diluents, lubricants, binders, granulating aids, colorants, flavorants and glidants that are conventional in the pharmaceutical art.

In order to facilitate the preparation of a solid, sustained release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and opioid or an opioid salt; (b) mixing the hydroxyalkyl cellulose containing granules with at least one C₁₂-C₃₆ aliphatic alcohol; and (c) optionally, compressing and shaping the granules. Preferably, the granules are formed by wet granulating the hydroxyalkyl cellulose/opioid with water. In a particularly preferred embodiment of this process, the amount of water added during the wet granulation step is preferably between 1.5 and 5 times, especially between 1.75 and 3.5 times, the dry weight of the opioid.

In yet other alternative embodiments, a spheronizing agent, together with the active ingredient can be spheronized to form spheroids. Microcrystalline cellulose is preferred. A suitable microcrystalline cellulose is, for example, the material sold as Avicel PH 101 (Trade Mark, FMC Corporation). In such embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxypropylcellulose, are preferred. Additionally (or alternatively) the spheroids may contain a water insoluble polymer, especially an acrylic polymer, an acrylic copolymer, such as a methacrylic acid-ethyl acrylate co-polymer, or ethyl cellulose. In such embodiments, the sustained release coating will generally include a hydrophobic material such as (a) a wax, either alone or in admixture with a fatty alcohol; or (b) shellac or zein.

### Melt Extrusion Matrices

In certain preferred embodiments of the present invention, the sustained release matrices also be prepared via melt-granulation or melt-extrusion techniques. Such formulations are described in U.S. patent application Ser. No. 08/334,209, filed Nov. 4, 1994 and U.S. patent application Ser. No. 08/833,948, filed Apr. 10, 1997, both of which are hereby incorporated by reference in their entireties. Generally, melt-granulation techniques involve melting a normally solid hydrophobic material, e.g. a wax, and incorporating a powdered drug therein. To obtain a sustained release dosage form, it may be necessary to incorporate an additional hydrophobic substance, e.g. ethylcellulose or a water-insoluble acrylic polymer, into the molten wax hydrophobic material. Examples of sustained release formulations prepared via melt-granulation techniques are found in U.S. Pat. No. 4,861,598, assigned to the Assignee of the present invention and hereby incorporated by reference in its entirety.

The additional hydrophobic material may comprise one or more water-insoluble wax-like thermoplastic substances possibly mixed with one or more wax-like thermoplastic substances being less hydrophobic than said one or more water-insoluble wax-like substances. In order to achieve constant release, the individual wax-like substances in the formulation should be substantially non-degradable and insoluble in gastrointestinal fluids during the initial release phases. Useful water-insoluble wax-like substances may be those with a water-solubility that is lower than about 1:5,000 (w/w).

In addition to the above ingredients, a sustained release matrix may also contain suitable quantities of other materials, e.g., diluents, lubricants, binders, granulating aids, colorants, flavorants and glidants that are conventional in the pharmaceutical art. The quantities of these additional materials will be sufficient to provide the desired effect to the desired formulation. In addition to the above ingredients, a sustained release matrix incorporating melt-extruded multiparticulates may also contain suitable quantities of other materials, e.g. diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986), incorporated by reference herein.

The preparation of a suitable melt-extruded matrix according to the present invention may, for example, include the steps of blending the opioid analgesic (i.e., hydromorphone) together with at least one hydrophobic material and preferably the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides sustained release of the therapeutically active agent for a time period of from about 8 to about 24 hours. The multiparticulates may be divided into unit doses via placement into a gelatin capsule, or may be compressed into a suitable tablet form.

An optional process for preparing the melt extrusions of the present invention includes directly metering into an extruder a hydrophobic material, a therapeutically active agent, and an optional binder; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one preferred embodiment, oral dosage forms are prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

In yet another preferred embodiment, the extrudate can be shaped into tablets as set forth in U.S. Pat. No. 4,957,681 (Klimesch, et. al.), described in additional detail above and hereby incorporated by reference.

Optionally, the sustained release melt-extruded multiparticulate systems or tablets can be coated, or the gelatin capsule can be further coated, with a sustained release coating such as the sustained release coatings described above. Such coatings preferably include a sufficient amount of hydrophobic material to obtain a weight gain level from about 2 to about 30 percent, although the overcoat may be greater depending upon the physical properties of the particular opioid analgesic compound utilized and the desired release rate, among other things.

The melt-extruded unit dosage forms of the present invention may further include combinations of melt-extruded multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated, e.g., as separate pellets within a gelatin capsule, or may be coated on the surface of the multiparticulates after preparation of the dosage forms (e.g., controlled release coating or matrix-based). The unit dosage forms of the present invention may also contain a combination of controlled release beads and matrix multiparticulates to achieve a desired effect.

The sustained release formulations of the present invention preferably slowly release the therapeutically active agent, e.g., when ingested and exposed to gastric fluids, and then to intestinal fluids. The sustained release profile of the melt-extruded formulations of the invention can be altered, for example, by varying the amount of retardant, i.e., hydrophobic material, by varying the amount of plasticizer relative to hydrophobic material, by the inclusion of additional ingredients or excipients, by altering the method of manufacture, etc.

In other embodiments of the invention, the melt extruded material is prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/ or the retardant material.

### DETAILED DESCRIPTION OF PREFERRED EMBODIMENTS

The following examples illustrate various aspects of the present invention. They are not to be construed to limit the claims in any manner whatsoever.

### MELT-EXTRUSION TECHNIQUES

Typical melt extrusion systems capable of carrying-out the present invention include a suitable extruder drive motor having variable speed, torque control, and start-stop controls. In addition, the system will include a temperature control console which includes temperature sensors, cooling means and temperature indicators throughout the length of the extruder. In addition, the system will include an extruder such as a single-screw extruder, twin-extruder which consists of two counter-rotating intermeshing screws enclosed within a cylinder or barrel having an aperture or die at the exist thereof. The feed materials enter through a feed hopper and is moved through the barrel by the screws and is forced through the die into strands which are thereafter conveyed such as by a continuous movable belt to allow for cooling and being directed to a pelletizer or other suitable device to render the extruded ropes into a multiparticulate system. The pelletizer can consist of rollers, fixed knife, rotating cutter and the like. Suitable instruments and systems are available from distributors such as C.W. Brabender Instruments, Inc. of South Hackensack, N.J., or Leistritz of Somerville, N.J. Other suitable apparatus will be apparent to those of ordinary skill in the art.

The melt extruded product is preferably prepared in a manner which substantially excludes air during the extrusion phase of the process. This may be accomplished, for example, by using a Leistritz extruder having a vacuum attachment. It has been found that extruded multiparticulates prepared according to the invention using the Leistritz extruder under vacuum provides a melt-extruded product having different physical characteristics. In particular, the extrudate is substantially non-porous when magnified, e.g., using a scanning electron microscope which provides an SEM (scanning electron micrograph). It has been found that such substantially non-porous formulations provide a faster release of the therapeutically active agent, relative to the same formulation prepared without vacuum. SEMs of the multiparticulates prepared using an extruder under vacuum appear very smooth, and the multiparticulates tend to be more robust than those multiparticulates prepared without vacuum.

A general procedure for preparing melt extrusion formulations useful in the present invention is as follows. First, the required amount of drug (hydromorphone), hydrophobic material and binder are blended, along with any additional excipients. Next, a powder feeder is charged with the proper amount of drug/excipient blend. The temperatures of the extruder heating zones are set to the required temperature, depending on the formulation. Typically, in the Leistritz extruder, the temperatures should be set from about 70° to about 105° C. The blend is preferably not fed into the extruder until the corresponding heating zones reach steady temperatures. Preferably, the extruder screw rotation speed may be set to, e.g., about 200 rpm, and the feeder, the conveyor and the pelletizer are actuated. After the excipients in the blend are (preferably) softened and the drug is embedded in the softened mixture, the resultant viscous mass is extruded as spaghetti-like strands. The diameter of the extruder aperture can be adjusted to vary the thickness of the resulting strand. The conveyor belt speed may be set to an appropriate speed (e.g., 3-100 ft/min). The extruded semisolid strand(s) are allowed to congeal and/or harden while transported to the pelletizer on the conveyor belt. Additional cooling devices may be needed to ensure proper congealing (the conveyor belt may not be needed to cool the strand, if the material congeals rapidly enough). The roller knife is preferably set to an appropriate speed (e.g., to 3-100 ft/min and 100-800 rpm), and the congealed strands are preferably cut to desired size (e.g., 0.5-5 mm in diameter, e.g., 0.3.5 mm in length), The product, in the form of pellets, is then collected. A desired weight of pellets may then be filled into hard gelatin capsules to obtain an appropriate dose of the drug or, alternatively, the pellets can be milled and compressed into tablets.

### Dissolution Method

The following dissolution method was used to obtain dissolution profiles for the dosage forms of the Examples: (USP II Paddle at 100 rpm at 37° C.). The media is as follows: 1st hour in 700 ml simulated gastric fluid (SGF), pH 1.2 without enzyme, and thereafter, 900 ml simulated intestinal fluid (SIF), pH 7.5 without enzyme. HPLC procedures may be used for assaying.

### EXAMPLES 1-2

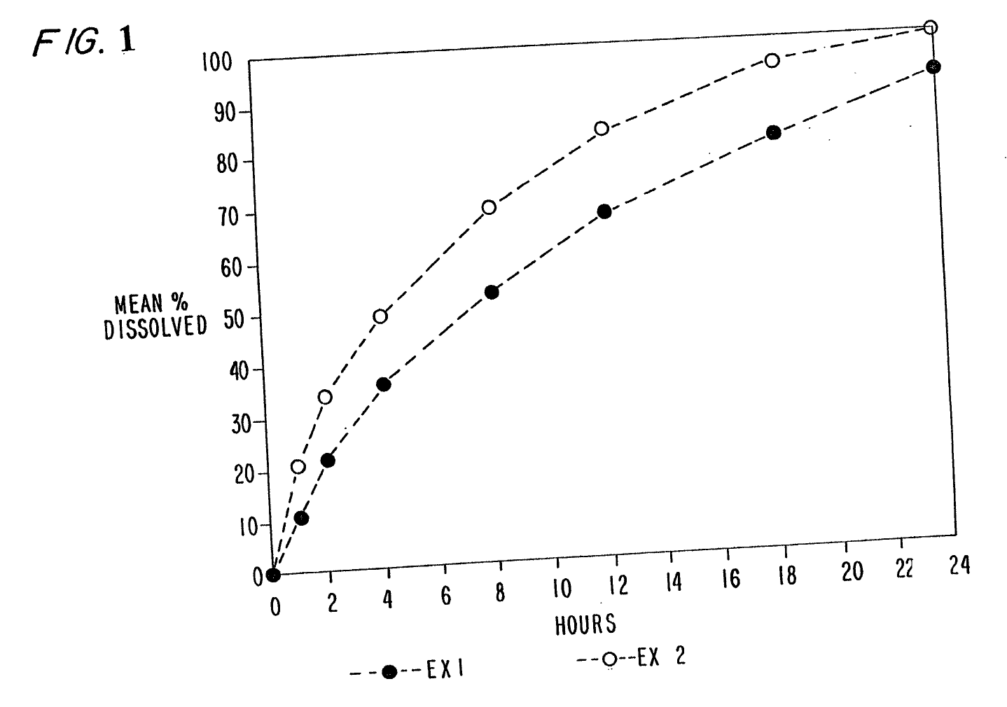

In Examples 1-2, hydromorphone hydrochloride controlled release pellets were prepared according to the above manufacturing procedure using Eudragit RSPO and Eudragit L-100 as the retardants. The formulations are set forth in Tables 1 and 2 below. The dissolution of these formulations is set forth in FIG. 1.

TABLE 1

| Ex.1 - Composition | Amt. (mg) per capsule |
|---|---|
| Hydromorphone Hydrochloride | 8 |
| Eudragit RSPO | 67.2 |
| Eudragit L-l00 | 11.2 |
| Stearic Acid | 33.6 |
| Total | 120 |

TABLE 2

| Ex. 2 - Composition | Amt. (mg) per capsule |
|---|---|
| Hydromorphone Hydrochloride | 8 |
| Eudragit RSPO | 36 |
| Eudragit L-100 | 6 |
| Stearic Acid | 18 |
| Talc | 52 |
| Total | 120 |

### EXAMPLE 3

Hydromorphone HCL once-a-day capsules were produced with the formula set forth in Table 3 below, using the melt extrusion methods and technology described above.

TABLE 3

| | | Percentage |
|---|---|---|
| Ingredients | Amt(mg)/Capsule | in Formula |
| Hydromorphone HCL | 8 | 10 |
| Eudragit RSPO | 53 | 66.25 |
| Stearyl Alcohol | 19 | 23.75 |
| Total | 80 | 100 |

Pellets of 1.0 mm in diameter and 1.0 mm in length were prepared. Each capsule holds 80 mg of pellets and contains 8 mg of hydromorphone HCL. The above capsules were tested using the dissolution methodology described above, and were found to have the dissolution results set forth in Table 4 below:

TABLE 4

| Time (hr) | 1 | 2 | 4 | 8 | 12 | 18 | 24 |
|---|---|---|---|---|---|---|---|
| Mean % dissolved | 17 | 28 | 32 | 45 | 56 | 69 | 82 |

### EXAMPLE 4

Hydromorphone HCL once-a-day capsules were produced with the formula set forth in Table 5 below:

TABLE 5

| | | Percentage |
|---|---|---|
| Ingredients | Amt(mg)/Capsule | in Formula |
| Hydromorphone HCL | 8 | 10 |
| Eudragit RSPO | 48 | 60 |
| Stearyl Alcohol | 24 | 30 |
| Total | 80 | 100 |

The pellet manufacturing procedure and the dissolution method are the same as described in Example 3.

The above capsules were found to have the dissolution results set forth in Table 6 below:

TABLE 6

| Time (hr) | 1 | 2 | 4 | 8 | 12 | 18 | 24 |
|---|---|---|---|---|---|---|---|
| Mean % dissolved | 23 | 29 | 40 | 56 | 69 | 84 | 96 |

### EXAMPLE 5

Hydromorphone HCL once-a-day capsules were produced with the following formula set forth in Table 7 according to the method described in Example 3.

TABLE 7

| | | Percentage |
|---|---|---|
| Ingredients | Amt(mg)/Capsule | in Formula |
| Hydromorphone HCL | 8 | 10 |
| Eudragit RSPO | 41.5 | 51.9 |
| Eudragit L-100 | 8.5 | 10.6 |
| Stearic Acid | 22 | 27.5 |
| Total | 80 | 100 |

The manufacturing procedure of the pellets and the dissolution method are the same as described in Example 3. The above capsules were found to have the following dissolution results set forth in Table 8:

TABLE 8

| Time (hr) | 1 | 2 | 4 | 8 | 12 | 18 | 24 |
|---|---|---|---|---|---|---|---|
| Mean % dissolved | 4 | 14 | 36 | 52 | 64 | 75 | 84 |

### EXAMPLE 6

A bioavailability study of hydromorphone capsules of Examples 3 and 4 was conducted using a single dose, five-way crossover study in 12 normal male volunteers. The subjects received either 8 mg of Dilaudid tablet (immediate release hydromorphone commercially available from Knoll) or 8 mg of sustained release hydromorphone capsules. Dilaudid tablets were administered after an overnight fast. Hydromorphone sustained release capsules were administered with or without food. Blood samples were taken periodically and assayed for hydromorphone concentrations using gas chromatography with mass detection (G/MS). From the data, the following pharmacokinetic parameters were calculated as set forth in Table 9 below:

TABLE 9

| | AUC, | | |
|---|---|---|---|
| Treatment | n. hr/ml | Cmax, n/ml | Tmax, hr |
| Example 3, fasted | 19.23 | 0.76 | 3.9 |
| Example 3, fed | 21.47 | 0.93 | 1.9 |
| Example 4, fasted | 19.00 | 0.72 | 6.8 |
| Example 4, fed | 20.10 | 0.75 | 2.4 |
| Dilaudid, fasted | 14.55 | 3.69 | 0.7 |

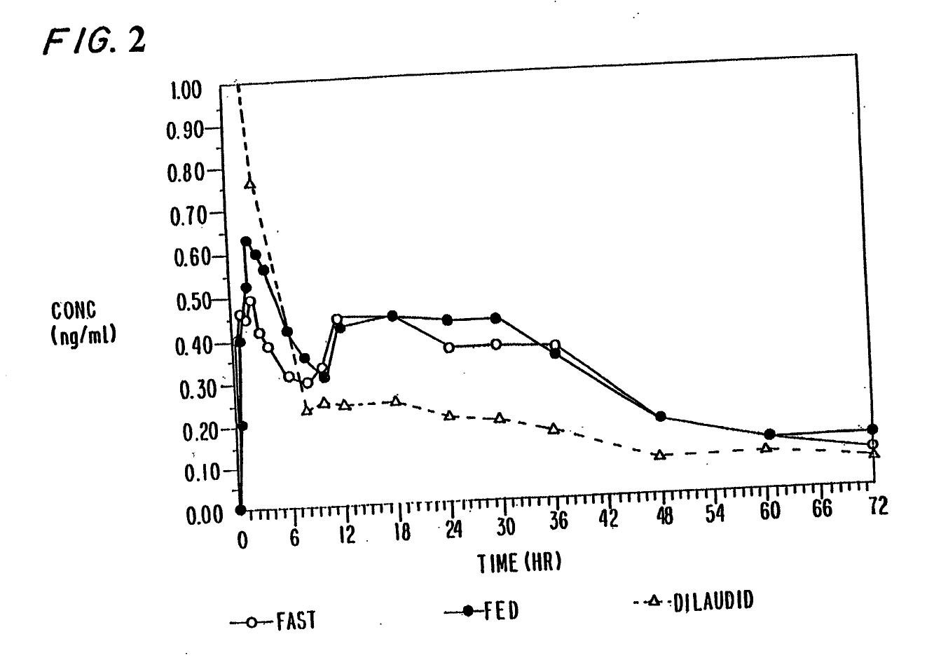

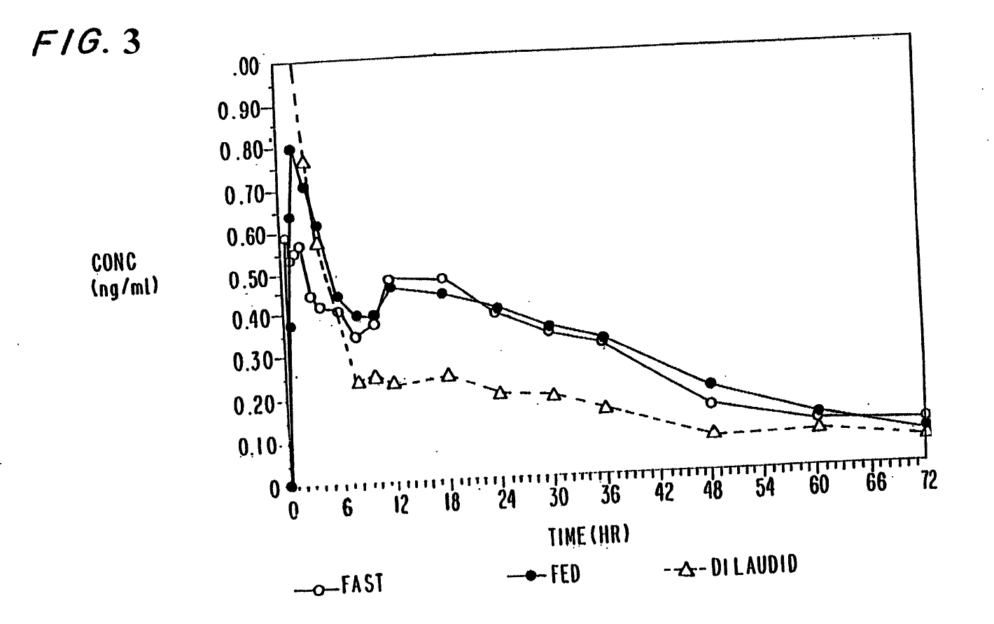

From the above data, both formulations 3 and 4 would be suitable for once-a-day administration both not having a food effect. The data of Example 3 is shown graphically in FIG. 2 and the data of Example 4 is shown graphically in FIG. 3.

### EXAMPLE 7

To assess steady state plasma levels and the effect of food on hydromorphone, a single dos, two-way crossover study was conducted in 12 normal male volunteers. The subjects received either 4 mg of Dilaudid (immediate release) every 6 hours or 16 mg of the capsules according to Example 3 every 24 hours. Venous blood samples were taken at predetermined time points. The plasma hydromorphone concentrations were quantitated using gas chromatography with mass detection (G/MS). From the data from day 4, the following pharmacokinetic parameters were calculated and are set forth in Table 10 below.

TABLE 10

| | AUC, | Cmax, | | |
|---|---|---|---|---|
| Treatment | n. hr/ml | n/ml | Cmin, n/ml | Tmax, hr |
| Example 16 | 36.08 | 2.15 | 1.49 | 5.8 |
| Dilaudid | 33.53 | 3.44 | 0.94 | 1.6 |

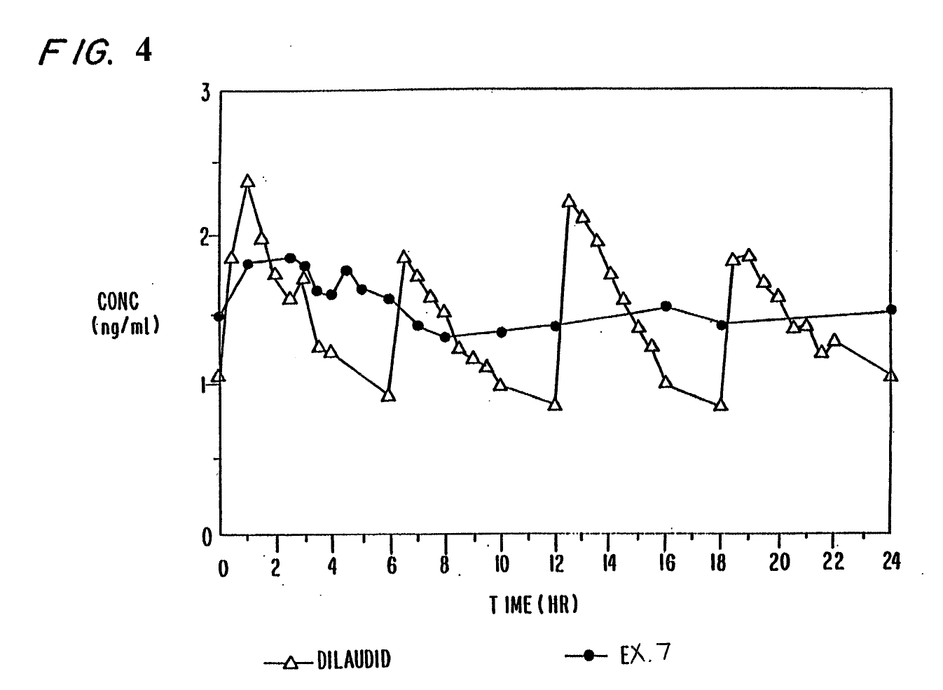

The results are shown graphically in FIG. 4. From this data it can be seen that Example 3 is suitable for once-a-day administration for either single dose or multiple dose administration.

### EXAMPLE 8

To assess bioavailability and effect of food on hydromorphone sustained release capsules, a single dose, three-way crossover study was conducted in 12 normal male volunteers. The subjects received either 8 mg of Dilaudid tablet (immediate release) or 8 mg of hydromorphone sustained release capsules of Example 5. Dilaudid tablets were administered after an overnight fast. The capsules of Example 5 were administered with or without food. Venous blood samples were taken at predetermined at time points. The plasma hydromorphone concentrations were quantitated using gas chromatography with mass detection (GC/MS).

From the data, the following pharrnacokinetic parameters were calculated and are set forth in Table 11 below:

TABLE 11

| | AUC, | Cmax, | |
|---|---|---|---|
| Treatment | n. hr/ml | n/ml | Tmax, hr |
| Example 5, fasted | 15.83 | 0.52 | 5.6 |
| Example 5, fed | 16.55 | 0.65 | 4.1 |
| Diludid, fasted | 16.54 | 3.15 | 0.8 |

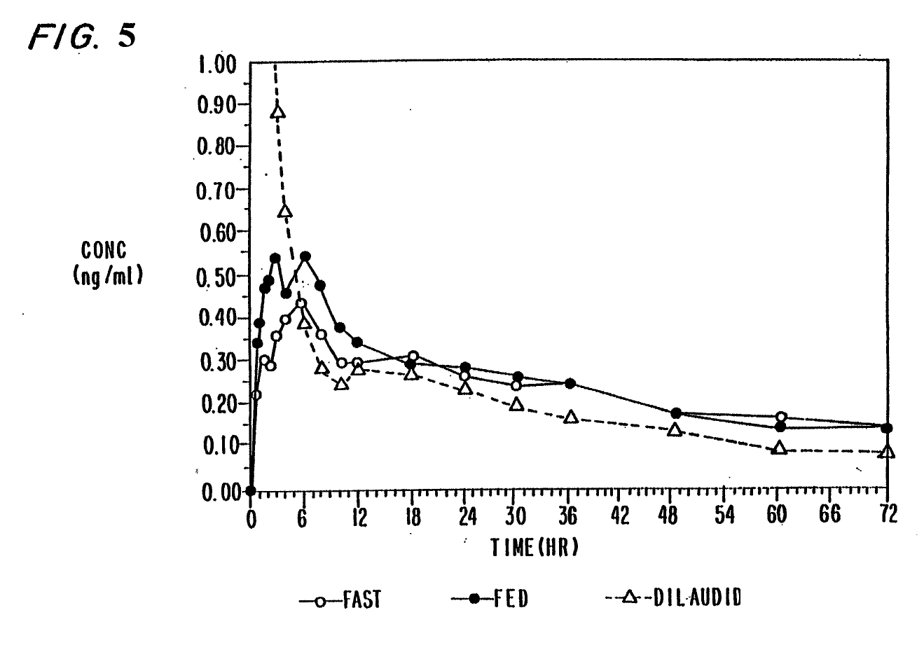

From the above data it can be concluded that a once-a-day hydromorphone product can be produced using other ingredients that are used for Examples 3 and 4. This data is shown graphically in FIG. 5.

### REVIEW AND COMMENT REGARDING WHETHER DOUBLE-HUMPED OR NOT

### Examples 9-10

Hydromorphone HCL once-a-day capsules were produced with the following formula set forth in Table 12 (Example 9) and Table 13 (Example 10) according to the method described in Example 3. The pellets were prepared by first mixing the Hydromorphone HCI with the retardant polymers. Then, using a twin screw melt extruder (Leistritz), the material was heated and extruded into 1 mm diameter “spaghetti like” extrudate. This extrudate was cooled on a conveyor belt and cut into 1 mm length pellets using a rotating knife pelletizer. These pellets were then collected and filled in capsules using a capsule filling machine at the appropriate fill weights to produce various strength capsules.

TABLE 12

| Ex. 9 - INGREDIENT | AMOUNT/UNIT |
|---|---|
| Hydromorphone HCI | 12.0 mg |
| Eudragit RSPO | 76.5 mg |
| Ethocel Std. 7 Premium | 4.5 mg |
| Stearyl Alcohol | 27.0 mg |
| Total | 120.0 mg |

TABLE 13

| Ex. 10 - INGREDIENT | AMOUNT/UNIT |
|---|---|
| Hydromorphone HCI | 24.0 mg |
| Eudragit RSPO | 153.5 mg |
| Ethocel Std. 7 Premium | 9.0 mg |
| Stearyl Alcohol | 54.0 mg |
| Total | 240.0 mg |

The capsules of Examples 9 and 10 were subjected to dissolution according to the following methodology—900 ml of simulated intestinal fluid (SIF)(USP) having 3 grams/liter of NaCl at pH 7.5. The results are set forth in Table 14 below:

TABLE 14

| Time (hr) | 1 | 2 | 4 | 6 | 12 | 18 | 24 |
|---|---|---|---|---|---|---|---|
| Ex. 9 | 11.3 | 18.2 | 31.8 | 56.0 | 73.8 | 90.3 | 98.9 |
| Ex. 10 | 11.1 | 18.3 | 33.3 | 58.2 | 77.0 | 92.6 | 98.9 |

### EXAMPLE 11

In Example 11, a four-treatment, randomized, crossover, analytically blinded single dose study (fed and fasted) was conducted in normal human volunteers. The dosage forms administered were a 12 mg sustained release hydromorphone capsule corresponding to Example 9 (referred to as “HHCR” 12 mg); a 24 mg sustained release hydromorphone capsule corresponding to Example 10 (referred to as “HHCR” 24 mg); and Dilaudid 8 mg tablets. The objective was to assess the pharmacokinetic and pharmacodynamic profile of HHCR 24 mg fed and fasted, HHCR fasted, and Dilaudid fasted. The sample size was 28 normal adult males (75 -100 KG) to complete 24. Blood samples were taken from the volunteers at times 0; 0.25; 0.5; 1; 1.5; 2; 3; 4; 5; 6; 8; 10; 12; 14; 18; 24; 36; 48; 60 and 72 hours. The assay used was a validated GC/MS for hydromorphone.

Table 15 is summary of adjusted hydromorphone pharmacokinetic parameters for HHCR 24 mg fed, HHCR 24 mg fasted, HHCR 12 mg fasted and Dilaudid 8 mg fasted.

TABLE 15

| | | Arithmetic Mean | | Geometric |
|---|---|---|---|---|
| Treatment | Parameter | (SD) | | Mean |
| HHCR 24 mg | | | | |
| Fasted | | | | |
| | AUC (0-Last) | 48.66 | (13.95) | 40.88 |
| | Cmax (ng/ml) | 1.27 | (0.37) | 1.22 |
| | T ½ Abs. (hrs) | 10.91 | (10.18) | |
| | T ½ Elim. (hrs) | 18.45 | (17.18) | |
| | Tmax (hrs) | 21.08 | (9.72) | |
| | Peak Width @ | 38.27 | (9.48) | |
| | 50% Cmax | | | |
| | Wag-Nel 50% | 24.71 | (2.29) | |
| | (hrs) | | | |
| HHCR 24 mg | | | | |
| Fed | | | | |
| | AUC (0-Last) | 48.36 | (12.60) | 44.88 |
| | Cmax (ng/ml) | 1.48 | (0.49) | 1.41 |
| | T ½ Abs. (hrs) | 3.92 | (4.45) | |
| | T ½ Elim. (hrs) | 26.98 | (14.03) | |
| | Tmax (hrs) | 8.54 | (0.95) | |
| | Peak Width @ | 26.87 | (13.65) | |
| | 50% Cmax | | | |
| | Wag-Nel 50% | 23.99 | (2.84) | |
| | (hrs) | | | |
| HHCR 12 mg | | | | |
| Fasted | | | | |
| | AUC (0-Last) | 43.14 | (14.07) | 41.15 |
| | Cmax (ng/ml) | 1.50 | (0.50) | 1.49 |
| | T ½ Abs. (hrs) | 8.08 | (8.34) | |
| | T ½ Elim. (hrs) | 20.14 | (16.69) | |
| | Tmax (hrs) | 16.00 | (8.82) | |
| | Peak Width @ | 26.72 | (8.24) | |
| | 50% Cmax | | | |
| | Wag-Nel 50% | 21.02 | (3.77) | |
| | (hrs) | | | |
| Dilaudid 8 mg | | | | |
| Fasted | | | | |
| | AUC (0-Last) | 32.26 | (12.23) | 28.87 |
| | Cmax (ng/ml) | 8.36 | (3.88) | 7.79 |
| | T ½ Abs. (hrs) | 0.78 | (0.41) | |
| | T ½ Elim. (hrs) | 1.04 | (0.40) | |
| | Tmax (hrs) | 0.79 | (0.29) | |
| | Peak Width @ | 1.21 | (0.40) | |
| | 50% Cmax | | | |
| | Wag-Nel 50% | 6.84 | (5.51) | |
| | (hrs) | | | |

Note:
Concentrations were dose-adjusted to 24 mg, only the AUC and Cmax are affected. Wag-Nel denotes “Wagner-Nelson”.

Table 16 is a summary of the adjusted hydromorphone pharmacokinetic parameters for HHCR 24 mg fed, HHCR 24 mg fasted, HHCR 12 mg fasted, and Dilaudid 8 mg fasted.

TABLE 16

| | | | 90% CI | |
|---|---|---|---|---|
| Parameter | | Ratio (%)* | Lower | Upper |
| HHCR 24 mg Fed vs. | | | | |
| HHCR 24 mg Fasted | | | | |
| AUC(0-Last) | Arithmetic | 95.31 | 88.12 | 102.88 |
| | Geometric | 96.29 | 87.38 | 106.12 |
| Cmax(ng/ml) | Arithmetic | 116.87 | 50.90 | 186.68 |
| | Geometric | 116.01 | 106.37 | 128.49 |
| T ½ Abs. (hrs) | Arithmetic | 35.94 | 6.27 | 65.33 |
| T ½ Elim. (hrs) | Arithmetic | 138.72 | 105.44 | 170.03 |
| Tmax (hrs) | Arithmetic | 40.56 | 21.61 | 60.49 |
| Peak Width @ 50% | Arithmetic | 67.61 | 56.81 | 79.19 |
| Cmax | | | | |
| Wag-Ne1 50% (hrs) | Arithmetic | 97.10 | 89.70 | 102.88 |
| HHCR 24 mg Fasted | | | | |
| vs. | | | | |
| Dilaudid 8 mg Fasted | | | | |
| AUC(0-Last) | Arithmetic | 150.83 | 130.40 | 161.67 |
| | Geometric | 155.31 | 140.93 | 171.18 |
| Cmax(ng/ml) | Arithmetic | 16.18 | 4.81 | 25.14 |
| | Geometric | 15.47 | 14.08 | 17.01 |
| T ½ Abs. (hrs) | Arithmetic | 1397.16 | 1143.4 | 2124.7 |
| T ½ Elim. (hrs) | Arithmetic | 1875.84 | 1028.3 | 2008.9 |
| Tmax (hrs) | Arithmetic | 2680.53 | 3648.0 | 5404.4 |
| Peak Width @ 50% | Arithmetic | 3160.93 | 2677.8 | 3352.5 |
| Cmax | | | | |
| Wag-Ne1 50% (hrs) | Arithmetic | 381.24 | 346.31 | 385.21 |
| HHCR 12 mg Fasted | | | | |
| vs. | | | | |
| HHCR 24 mg Fasted | | | | |
| AUC(0-Last) | Arithmetic | 88.66 | 82.10 | 86.84 |
| | Geometric | 89.53 | 61.24 | 88.67 |
| Cmax(ng/ml) | Arithmetic | 118.19 | 53.12 | 188.81 |
| | Geometric | 118.02 | 108.29 | 130.81 |
| T ½ Abs. (hrs) | Arithmetic | 74.06 | 44.25 | 104.30 |
| T ½ Elim. (hrs) | Arithmetic | 103.57 | 72.90 | 137.49 |
| Tmax (hrs) | Arithmetic | 76.26 | 56.17 | 95.08 |
| Peak Width @ 50% | Arithmetic | 88.82 | 58.58 | 80.84 |
| Cmax | | | | |
| Wag-Ne1 50% (hrs) | Arithmetic | 95.71 | 81.62 | 94.81 |
| HHCR 12 mg Fasted | | | | |
| vs. | | | | |
| Dilaudid 8 mg Fasted | | | | |
| AUC(0-Last) | Arithmetic | 133.73 | 123.08 | 148.73 |
| | Geometric | 139.06 | 128.18 | 163.24 |
| Cmax(ng/ml) | Arithmetic | 17.84 | 7.88 | 28.28 |
| | Geometric | 18.42 | 10.76 | 20.24 |
| T ½ Abs. (hrs) | Arithmetic | 1034.74 | 729.05 | 1704.3 |
| T ½ Elim. (hrs) | Arithmetic | 1840.75 | 1107.2 | 2088.2 |
| Tmax (hrs) | Arithmetic | 2028.85 | 2541.5 | 4301.0 |
| Peak Width @ 50% | Arithmetic | 2208.87 | 1765.8 | 2440.5 |
| Cmax | | | | |
| Wag-Ne1 50% (hrs) | Arithmetic | 320.44 | 302.83 | 851.53 |

Note:
Ratio and 90% CI are based on least squares means
*Ratio (%): (Test mean/Reference mean) 100%
Note:
Concentrations were dose-adjusted to 24 mg, only the AUC and Cmax are affected.
Note:
Wag-Ne1 denotes “Wagner-Nelson”

Table 17 provides a summary of the adjusted plasma hydromorphone concentration (ng/ml) at each sampling time by subject for HHCR 24 mg Fed. Table 18 provides a summary of the adjusted plasma hydromorphone concentration (ng/ml) at each sampling time by subject for HHCR 24 mg Fasted. Table 19 provides a summary of the adjusted plasma hydromorphone concentration (ng/ml) at each sampling time by subject for HHCR 12 mg Fasted. Table 20 provides a summary of the adjusted plasma hydromorphone concentration (ng/ml) at each sampling time by subject for Dilaudid 8 mg Fasted.

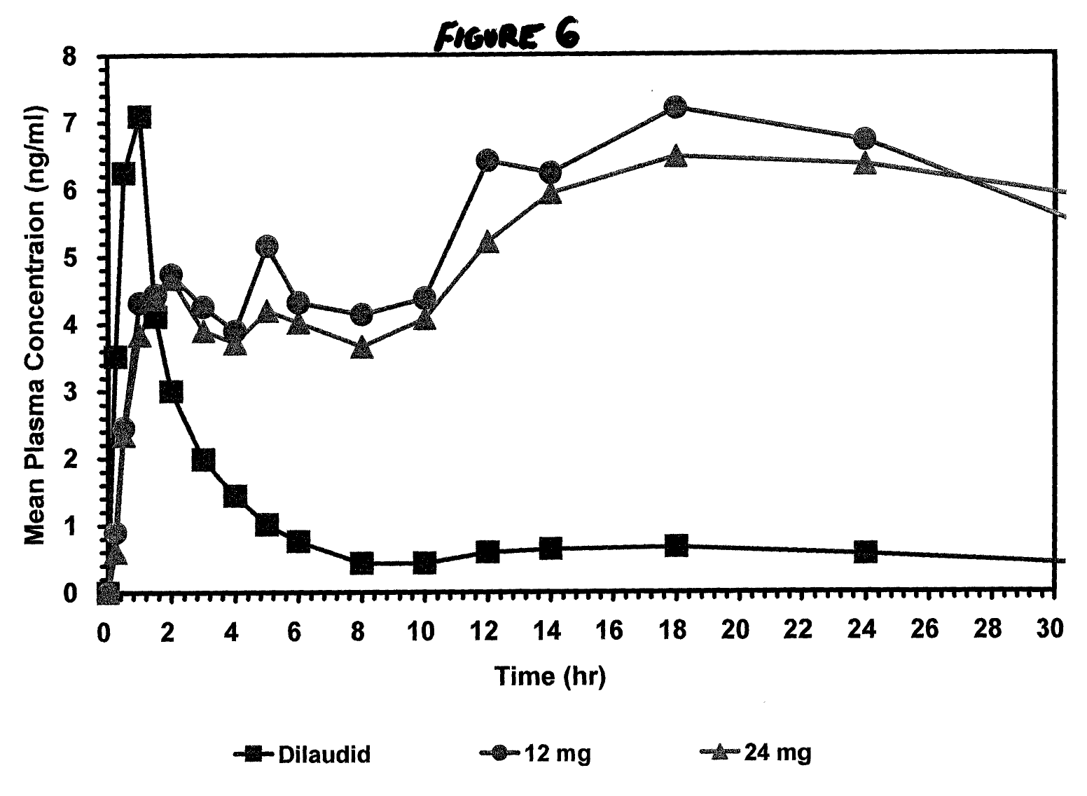

The results of this single-dose study show that the 24 mg capsule was dose proportional to the 12 mg capsule based on primary pharmacokinetic metrics. FIG. 6 provides the mean plasma concentration over time (up to 30 hours) for HHCR 12 mg, HHCR 24 mg, and Dilaudid.

TABLE 17
SUMMARY OF THE ADJUSTED PLASMA HYDROMORPHOYE CONCENTRATION (NG/ML) AT EACH SAMPLING TIME BY SUBJECT

| | |
|---|---|
| SUBJECT | TREATMENT: HHCR 24 mg Fed |

| NUMBER | 0 | 0.25 | 0.5 | 1 | 1.5 | 2 | 3 | 4 | 5 | 6 | 8 | 10 | 12 | 14 | 18 | 24 | 36 | 48 | 60 | 72 |
|---|---|---|---|---|---|---|---|---|---|---|---|---|---|---|---|---|---|---|---|---|
| 1 | 0.00 | 0.38 | 0.56 | 2.08 | 1.85 | 1.88 | 1.38 | 1.09 | 0.90 | 0.78 | 0.82 | 0.98 | 1.15 | 1.29 | 1.41 | 1.07 | 0.90 | 0.32 | 0.18 | 0.00 |
| 3 | 0.00 | 0.00 | 0.14 | 0.39 | 0.83 | 0.82 | 0.52 | 0.25 | 0.32 | 0.38 | 0.31 | 0.48 | 0.45 | 0.29 | 0.22 | 0.44 | 0.98 | 0.28 | 0.82 | 0.13 |
| 4 | 0.00 | 0.00 | 0.15 | 0.31 | 0.72 | 0.68 | 0.63 | 0.76 | 0.82 | 0.62 | 0.49 | 0.52 | 0.75 | 1.02 | 0.95 | 0.99 | 0.83 | 0.50 | 0.18 | 0.10 |
| 5 | 0.00 | 0.13 | 0.21 | 0.79 | 1.16 | 1.31 | 1.48 | 1.23 | 1.19 | 1.01 | 0.92 | 0.78 | 0.90 | 0.95 | 1.04 | 1.54 | 1.40 | 0.78 | 0.49 | 0.26 |
| 6 | 0.00 | 0.00 | 0.22 | 0.43 | 0.55 | 0.74 | 1.11 | 0.60 | 0.71 | 0.37 | 1.01 | 0.67 | 0.89 | 0.51 | 0.36 | 0.39 | 0.84 | 0.82 | 0.23 | 0.00 |
| 7 | 0.00 | 0.00 | 0.14 | 0.80 | 1.22 | 1.26 | 1.08 | 0.79 | 1.17 | 1.03 | 0.69 | 0.58 | 0.50 | 0.49 | 0.98 | 0.88 | 0.75 | 0.41 | 0.29 | 0.25 |
| 8 | 0.00 | 0.00 | 0.17 | 0.22 | 0.72 | 0.68 | 1.20 | 0.82 | 0.86 | 0.76 | 1.12 | 0.67 | 0.69 | 0.67 | 0.98 | 0.73 | 0.98 | 0.56 | 0.28 | 0.21 |
| 9 | 0.00 | 0.00 | 0.19 | 0.37 | 0.78 | 1.09 | 0.31 | 0.77 | 0.47 | 0.65 | 0.53 | 0.26 | 0.45 | 0.23 | 0.52 | 0.70 | 0.98 | 0.25 | 0.18 | 0.00 |
| 10 | 0.00 | 0.00 | 0.00 | 0.11 | 0.89 | 0.96 | 1.30 | 1.72 | 1.82 | 1.10 | 0.97 | 0.98 | 0.93 | 0.82 | 1.29 | 1.17 | 1.41 | 0.47 | 0.41 | 0.20 |
| 11 | 0.00 | 0.21 | 0.44 | 0.72 | 1.14 | 1.15 | 0.82 | 1.11 | 1.04 | 0.96 | 0.89 | 0.74 | 0.45 | 0.58 | 0.76 | 1.42 | 1.17 | 0.48 | 0.26 | 0.17 |
| 12 | 0.00 | 0.00 | 0.18 | 0.58 | 0.72 | 1.09 | 2.21 | 1.40 | 1.00 | 0.71 | 0.54 | 0.88 | 0.85 | 1.38 | 1.21 | 0.94 | 0.48 | 0.14 | 0.00 | 0.00 |
| 13 | 0.00 | 0.00 | 0.00 | 0.28 | 0.30 | 0.73 | 0.57 | 0.77 | 1.11 | 0.78 | 0.64 | 0.54 | 0.38 | 0.43 | 0.73 | 1.17 | 0.82 | 0.28 | 0.18 | 0.00 |
| 14 | 0.00 | 0.00 | 0.00 | 0.80 | 0.49 | 1.22 | 1.18 | 1.51 | 1.38 | 1.19 | 1.03 | 0.79 | 0.99 | 0.85 | 0.97 | 1.17 | 1.17 | 0.48 | 0.37 | 0.27 |
| 15 | 0.00 | 0.00 | 0.00 | 0.00 | 0.00 | 0.10 | 1.25 | 2.12 | 1.46 | 1.35 | 0.85 | 0.85 | 1.03 | 0.80 | 0.82 | 0.84 | 1.01 | 0.65 | 0.40 | 0.22 |
| 16 | 0.00 | 0.00 | 0.00 | 0.45 | 1.68 | 2.04 | 2.76 | 2.11 | 2.36 | 1.75 | 1.11 | 1.17 | 1.48 | 1.34 | 1.85 | 1.88 | 1.21 | 0.49 | 0.32 | 0.00 |
| 17 | 0.00 | 0.00 | 0.15 | 0.52 | 0.79 | 1.00 | 1.29 | 1.08 | 0.99 | 0.84 | 0.87 | 0.84 | 0.88 | 0.83 | 1.14 | 1.44 | 0.85 | 0.49 | 0.26 | 0.22 |
| 19 | 0.00 | 0.29 | 0.41 | 0.89 | 0.61 | 0.86 | 0.32 | 0.32 | 0.80 | 0.70 | 0.68 | 0.73 | 0.51 | 0.33 | 0.84 | 0.58 | 0.33 | 0.26 | 0.20 | 0.00 |
| 21 | 0.00 | 0.00 | 0.08 | 0.00 | 0.46 | 0.96 | 0.99 | 1.06 | 0.99 | 1.29 | 0.78 | 0.78 | 0.72 | 0.88 | 0.85 | 1.07 | 0.90 | 0.45 | 0.30 | 0.23 |
| 22 | 0.00 | 0.00 | 0.15 | 0.99 | 0.57 | 0.62 | 1.19 | 1.04 | 1.04 | 0.84 | 0.57 | 0.54 | 0.52 | 0.59 | 0.51 | 0.48 | 0.66 | 0.31 | 0.23 | 0.14 |
| 23 | 0.00 | 0.00 | 0.00 | 0.86 | 0.89 | 1.34 | 1.11 | 0.89 | 1.10 | 0.88 | 0.72 | 0.70 | 0.78 | 0.92 | 0.98 | 1.14 | 0.82 | 0.56 | 0.22 | 0.00 |
| 25 | 0.00 | 0.00 | 0.66 | 0.88 | 1.59 | 1.33 | 1.32 | 0.87 | 0.89 | 1.12 | 1.08 | 1.28 | 1.75 | 1.32 | 1.52 | 1.61 | 0.74 | 0.18 | 0.00 | 0.00 |
| 26 | 0.00 | 0.00 | 0.00 | 0.12 | 0.27 | 0.42 | 0.67 | 0.65 | 0.78 | 0.80 | 0.51 | 0.48 | 0.50 | 0.39 | 0.62 | 0.65 | 0.42 | 0.21 | 0.25 | 0.48 |
| 27 | 0.00 | 0.00 | 0.41 | 1.22 | 1.96 | 1.58 | 1.40 | 1.78 | 1.46 | 1.41 | 0.87 | 0.99 | 1.23 | 1.61 | 1.82 | 1.38 | 1.34 | 0.57 | 0.25 | 0.12 |
| 28 | 0.00 | 0.00 | 0.00 | 0.59 | 0.43 | 1.23 | 0.90 | 0.82 | 1.01 | 1.74 | 0.87 | 0.66 | 0.78 | 0.58 | 0.69 | 1.13 | 0.75 | 0.63 | 0.38 | 0.28 |
| H | 24 | 24 | 24 | 24 | 24 | 24 | 24 | 24 | 24 | 24 | 24 | 24 | 24 | 24 | 24 | 24 | 24 | 24 | 24 | 24 |
| Mean | 0.00 | 0.04 | 0.17 | 0.60 | 0.87 | 1.03 | 1.16 | 1.07 | 1.05 | 0.88 | 0.78 | 0.74 | 0.82 | 0.79 | 0.92 | 1.01 | 0.80 | 0.43 | 0.26 | 0.14 |
| Std. Dev | 0.00 | 0.10 | 0.18 | 0.45 | 0.53 | 0.44 | 0.51 | 0.50 | 0.40 | 0.38 | 0.22 | 0.23 | 0.34 | 0.35 | 0.39 | 0.36 | 0.29 | 0.17 | 0.11 | 0.18 |
| RSD % | 0.00 | 244.45 | 110.84 | 90.30 | 60.45 | 42.89 | 44.27 | 46.48 | 88.10 | 38.85 | 28.15 | 30.99 | 41.46 | 48.51 | 43.03 | 35.06 | 32.48 | 39.45 | 44.34 | 95.49 |

Note:
Concentrations were dose-adjusted in 24 mg.

TABLE 18
SUMMARY OF THE ADJUSTED PLASMA HYDROMORPHONE CONCENTRATION (NG/ML) AT EACH SAMPLING TIME BY SUBJECT

| SUBJECT | TREATMENT: HHCR 24 np Fasted | | | | | | | | | | | | | | | | | | | |
|---|---|---|---|---|---|---|---|---|---|---|---|---|---|---|---|---|---|---|---|---|
| NUMBER | 0 | 0.25 | 0.5 | 1 | 1.5 | 2 | 3 | 4 | 5 | 6 | 8 | 10 | 12 | 14 | 18 | 24 | 36 | 46 | 60 | 72 |
| 1 | 0.00 | 0.00 | 0.43 | 0.54 | 0.59 | 0.58 | 0.55 | 0.51 | 0.75 | 0.68 | 0.61 | 0.81 | 1.42 | 1.32 | 1.60 | 0.98 | 0.82 | 0.37 | 0.00 | 0.00 |
| 3 | 0.00 | 0.00 | 0.53 | 0.69 | 0.60 | 0.54 | 0.38 | 0.38 | 0.41 | 0.35 | 0.35 | 0.59 | 0.67 | 0.69 | 0.72 | 0.74 | 0.76 | 0.42 | 0.53 | 0.27 |
| 4 | . | 0.51 | 0.56 | 0.65 | 0.50 | 0.42 | 0.48 | 0.48 | 0.58 | 0.44 | 0.52 | 0.43 | 0.65 | 0.71 | 0.68 | 1.09 | 1.19 | 0.57 | 0.27 | 0.20 |
| 5 | 0.00 | 0.35 | 0.42 | 0.59 | 0.52 | 0.72 | 0.41 | 0.54 | 0.67 | . | . | . | 0.98 | 1.15 | 1.26 | 0.91 | 0.81 | 0.63 | 0.31 | 0.19 |
| 6 | 0.00 | 0.00 | 0.27 | 0.40 | 0.84 | 0.66 | 0.60 | 0.85 | 0.68 | 0.68 | 0.54 | 0.70 | 0.70 | 0.82 | 0.68 | 0.72 | 0.65 | 0.53 | 0.26 | 0.11 |
| 7 | 0.00 | 0.00 | 0.00 | 0.51 | 0.62 | 0.87 | 0.30 | 0.87 | 0.71 | 0.78 | 0.68 | 0.65 | 0.65 | 0.93 | 1.05 | 0.76 | 0.66 | 0.43 | 0.27 | 0.17 |
| 8 | 0.00 | 0.00 | 0.56 | 0.65 | 0.64 | 0.68 | 0.89 | 0.42 | 0.65 | 0.45 | 0.60 | 0.58 | 0.83 | 1.41 | 0.91 | 1.04 | 0.75 | 0.53 | 0.30 | 0.22 |
| 9 | 0.00 | 0.00 | 0.52 | 0.84 | 0.76 | 0.62 | 0.67 | 0.57 | 0.56 | 0.76 | 0.47 | 0.52 | 0.71 | 0.84 | 0.78 | 0.96 | 1.20 | 0.57 | 0.41 | 0.23 |
| 10 | 0.00 | 0.00 | 0.16 | 0.79 | 0.97 | 0.94 | 0.77 | 0.76 | 0.82 | 0.69 | 1.13 | 0.80 | 0.81 | 1.19 | 1.59 | 1.18 | 1.30 | 0.50 | 0.50 | 0.16 |
| 11 | 0.00 | 0.00 | 0.65 | 0.87 | 0.67 | 1.01 | 1.03 | 0.81 | 1.08 | 1.18 | 0.81 | 0.77 | 0.68 | 0.69 | 1.59 | 1.55 | 0.98 | 0.73 | 0.29 | 0.15 |
| 12 | 0.00 | 0.00 | 0.00 | 0.43 | 0.25 | 0.75 | 0.32 | 0.30 | 0.54 | 0.41 | 0.87 | 0.58 | 0.68 | 0.74 | 0.80 | 0.66 | 0.45 | 0.16 | 0.00 | 0.00 |
| 13 | 0.00 | 0.00 | 0.29 | 0.47 | 0.69 | 1.05 | 0.67 | 0.02 | 0.73 | 0.89 | 0.51 | 0.84 | 0.78 | 1.17 | 1.31 | 1.26 | 1.07 | 0.58 | 0.39 | 0.15 |
| 14 | 0.00 | 0.00 | 0.00 | 0.21 | 0.41 | 0.48 | 0.35 | 0.34 | 0.86 | 0.37 | 0.25 | 0.27 | 0.46 | 0.58 | 0.72 | 0.87 | 0.70 | 0.81 | 0.26 | 0.14 |
| 15 | 0.00 | 0.10 | 0.34 | 0.99 | 1.47 | 1.49 | 1.19 | 1.13 | 1.21 | 0.98 | 0.60 | 1.05 | 0.98 | 1.06 | 0.97 | 1.40 | 1.17 | 0.85 | 0.71 | 0.41 |
| 16 | 0.00 | 1.02 | 1.11 | 1.20 | 1.49 | 1.48 | 1.18 | 1.14 | 0.81 | 1.18 | 1.24 | 1.08 | 1.50 | 1.56 | 1.14 | 1.31 | 1.62 | 1.54 | 0.63 | 0.21 |
| 17 | 0.00 | 0.00 | 0.33 | 0.49 | 0.58 | 0.44 | 0.52 | 0.69 | 1.10 | 0.68 | 0.78 | 1.27 | 1.10 | 1.10 | 1.20 | 1.40 | 1.02 | 0.68 | 0.31 | 0.15 |
| 19 | 0.00 | 0.00 | 0.17 | 0.43 | 0.48 | 0.78 | 0.65 | 0.67 | 0.21 | 0.28 | 0.09 | 0.35 | 0.36 | 0.78 | 0.84 | 0.70 | 0.41 | 0.38 | 0.28 | 0.21 |
| 21 | 0.00 | 0.10 | 0.77 | 0.64 | 0.71 | 0.69 | 0.65 | 0.54 | 0.64 | 0.67 | 0.74 | 0.77 | 0.89 | 0.81 | 1.05 | 1.10 | 0.74 | 0.53 | 0.22 | 0.18 |
| 22 | 0.00 | 0.21 | 0.49 | 0.66 | 0.81 | 0.63 | 0.58 | 0.51 | 0.65 | 0.64 | 0.53 | 0.50 | 1.01 | 1.65 | 1.02 | 1.01 | 0.81 | 0.37 | 0.24 | 0.15 |
| 23 | 0.00 | 0.00 | 0.13 | 0.55 | 0.72 | 0.75 | 0.60 | 0.52 | 0.59 | 0.54 | 0.49 | 0.64 | 0.85 | 0.85 | 0.97 | 0.98 | 0.68 | 0.42 | 0.17 | 0.12 |
| 25 | 0.00 | 0.00 | 0.54 | 0.76 | 0.61 | 0.54 | 0.26 | 0.89 | 0.78 | 0.89 | 0.97 | 0.93 | 1.28 | 1.12 | 1.28 | 1.07 | 0.73 | 0.28 | 0.13 | 0.00 |
| 26 | 0.00 | 0.00 | 0.50 | 0.82 | 0.91 | 0.79 | 0.82 | 0.81 | 1.02 | 0.85 | 0.62 | 0.85 | 0.72 | 0.68 | 0.82 | 1.15 | 0.88 | 0.55 | 0.40 | 0.29 |
| 27 | 0.00 | 0.00 | 0.21 | 0.96 | 0.87 | 0.90 | 0.63 | 0.74 | 0.83 | 0.79 | 0.65 | 0.71 | 1.20 | 1.81 | 2.39 | 1.76 | 1.39 | 0.60 | 0.25 | 0.14 |
| 28 | 0.00 | 0.00 | 0.41 | 0.53 | 0.68 | 1.02 | 0.73 | 0.52 | 0.65 | 0.62 | 0.57 | 0.29 | 0.68 | 0.58 | 0.56 | 0.86 | 1.09 | 0.42 | 0.20 | 0.13 |
| H | 23 | 24 | 24 | 24 | 24 | 24 | 24 | 24 | 24 | 23 | 23 | 23 | 24 | 24 | 24 | 24 | 24 | 24 | 24 | 24 |
| Mean | 0.00 | 0.10 | 0.39 | 0.64 | 0.78 | 0.78 | 0.65 | 0.62 | 0.70 | 0.67 | 0.61 | 0.68 | 0.87 | 0.98 | 1.08 | 1.08 | 0.82 | 0.54 | 0.30 | 0.17 |
| Std. Dev | 0.00 | 0.23 | 0.28 | 0.22 | 0.29 | 0.26 | 0.24 | 0.22 | 0.23 | 0.24 | 0.27 | 0.25 | 0.29 | 0.36 | 0.41 | 0.28 | 0.29 | 0.28 | 0.18 | 0.09 |
| RS0 % | 0.00 | 246.14 | 68.48 | 38.97 | 39.03 | 36.10 | 37.49 | 35.71 | 33.29 | 35.39 | 44.43 | 38.24 | 33.08 | 38.31 | 37.62 | 26.39 | 32.04 | 48.51 | 59.44 | 54.83 |

Note:
Concentrations were dose-adjusted to 24 ng.

TABLE 19
SUMMARY OF THE ADJUSTED PLASMA HYDROMORPHONE CONCENTRATION (NG/ML) AT EACH SAMpLING TIME BY SUBJECT

| SUBJECT | TREATMENT: HFDR 12 mg Fasted | | | | | | | | | | | | | | | | | | | |
|---|---|---|---|---|---|---|---|---|---|---|---|---|---|---|---|---|---|---|---|---|
| NUMBER | 0 | 0.25 | 0.5 | 1 | 1.5 | 2 | 3 | 4 | 5 | 6 | 8 | 10 | 12 | 14 | 18 | 24 | 38 | 48 | 60 | 72 |
| 1 | 0.00 | 0.00 | 0.38 | 0.52 | 0.82 | 0.58 | 0.78 | 0.78 | 1.00 | 0.78 | 1.12 | 1.62 | 1.40 | 1.34 | 1.18 | 0.99 | 0.32 | 0.00 | 0.00 | 0.00 |
| 3 | 0.00 | 0.00 | 0.64 | 0.46 | 0.60 | 1.16 | 0.38 | 0.38 | 0.48 | 0.40 | 0.46 | 0.44 | 0.38 | 0.54 | 0.94 | 0.80 | 0.84 | 0.42 | 0.28 | 0.00 |
| 4 | 0.00 | 0.00 | 0.00 | 0.00 | 0.42 | 0.44 | 0.24 | 0.28 | 0.72 | 0.00 | 0.22 | 0.34 | 0.78 | 0.48 | 0.88 | 0.56 | 0.56 | 0.22 | 0.00 | . |
| 5 | 0.00 | 0.00 | 0.34 | 1.10 | 0.94 | 0.74 | 0.66 | 0.60 | 1.28 | 0.76 | 0.96 | 0.96 | 1.18 | 1.18 | 1.32 | 2.64 | 1.56 | 0.68 | 0.45 | 0.32 |
| 6 | 0.00 | 0.00 | 0.00 | 0.24 | 0.42 | 0.62 | 0.62 | 0.60 | 0.68 | 0.62 | 0.58 | 0.58 | 0.64 | 0.42 | 0.72 | 0.88 | 1.04 | 0.60 | 0.24 | 0.00 |
| 7 | 0.00 | 0.00 | 0.00 | 0.46 | 0.64 | 0.60 | 0.32 | 0.24 | 0.28 | 0.00 | 0.26 | 0.00 | . | 0.44 | 0.56 | 0.46 | 0.94 | 0.24 | 0.00 | 0.00 |
| 8 | 0.00 | 0.36 | 0.74 | 0.88 | 1.12 | 1.18 | 0.86 | 0.86 | 1.28 | 1.00 | 0.98 | 0.70 | 1.24 | 1.60 | 1.90 | 1.80 | 0.96 | 1.02 | 0.48 | 0.28 |
| 9 | 0.00 | 0.00 | 0.00 | 1.32 | 0.62 | 0.74 | 0.84 | 0.76 | 0.70 | 0.72 | 0.58 | 0.48 | 0.65 | 0.85 | 1.32 | 1.04 | 0.88 | 0.38 | 0.24 | 0.00 |
| 10 | 0.00 | 0.00 | 0.30 | 0.40 | 0.66 | 0.42 | 0.40 | 0.36 | 0.66 | 0.58 | 0.58 | 0.84 | 0.80 | 1.04 | 1.04 | 1.26 | 1.09 | 0.50 | 0.82 | 0.00 |
| 11 | 0.00 | 0.84 | 0.74 | 1.80 | 1.46 | 1.32 | 1.42 | 0.98 | 1.12 | 0.00 | 0.70 | 1.10 | 1.60 | 1.26 | 1.88 | 1.50 | 0.74 | 0.84 | 0.00 | 0.00 |
| 12 | 0.00 | 0.00 | 0.86 | 0.66 | 0.56 | 0.70 | 0.46 | 0.74 | 1.14 | 0.84 | 0.74 | 0.84 | 1.14 | 1.02 | 1.18 | 0.80 | 0.40 | 0.00 | 0.00 | 0.00 |
| 13 | 0.00 | 0.00 | 0.44 | 0.52 | 0.42 | 0.44 | 0.60 | 0.48 | 0.90 | 1.02 | 0.48 | 0.48 | 1.12 | 1.18 | 1.68 | 1.18 | 0.62 | 0.38 | 0.00 | 0.00 |
| 14 | 0.00 | 0.00 | 0.00 | 0.52 | 0.78 | 0.86 | 0.88 | 0.76 | 0.78 | 0.74 | 0.54 | 0.52 | 0.70 | 0.82 | 0.86 | 1.12 | 0.84 | 0.54 | 0.48 | 0.00 |
| 15 | 0.00 | 0.58 | 0.72 | 1.78 | . | 1.62 | 1.20 | 0.84 | 1.18 | 0.80 | 0.68 | 0.84 | 1.28 | 1.40 | 1.22 | 1.34 | 0.82 | 0.62 | 0.30 | 0.00 |
| 16 | 0.00 | 0.00 | 0.68 | 0.68 | 1.04 | 1.04 | 0.82 | 0.76 | 0.78 | 0.48 | 0.78 | 0.65 | 2.74 | 1.68 | 1.58 | 1.24 | 0.94 | 0.62 | 0.00 | 0.00 |
| 17 | 0.00 | 0.28 | 0.22 | 0.80 | 0.52 | 0.58 | 0.36 | 0.64 | 0.68 | 0.70 | 0.00 | 0.88 | 1.14 | 1.08 | 1.30 | 0.80 | 0.78 | 0.46 | 0.30 | 0.00 |
| 19 | 0.00 | 0.00 | 0.60 | 0.74 | 0.88 | 0.68 | 0.72 | 0.58 | 0.58 | 0.41 | 0.65 | 0.86 | 0.80 | 0.70 | 1.18 | 0.82 | 0.58 | 0.28 | 0.00 | 0.00 |
| 21 | 0.00 | 0.00 | 0.00 | 0.82 | 0.50 | 0.38 | 0.36 | 0.50 | 0.48 | 0.31 | 0.28 | 0.56 | 0.98 | 0.90 | 1.26 | 0.78 | 0.42 | 0.30 | 0.00 | 0.00 |
| 22 | 0.00 | 0.40 | 0.66 | 0.78 | 0.64 | 0.76 | 0.84 | 0.88 | 1.22 | 1.38 | 0.83 | 0.88 | 0.98 | 1.56 | 1.02 | 0.94 | 0.84 | 0.48 | 0.00 | 0.00 |
| 23 | 0.00 | 0.22 | 0.88 | 0.88 | 1.48 | 1.40 | 0.78 | 0.74 | 0.62 | 0.68 | 0.48 | 0.86 | 0.75 | 0.80 | 1.04 | 1.14 | 0.88 | 0.78 | 0.42 | 0.24 |
| 25 | 0.00 | 0.00 | 0.55 | 0.84 | 0.58 | 0.84 | 0.58 | 0.40 | 1.04 | 1.03 | 1.02 | 0.84 | 1.34 | 1.18 | 0.88 | 0.72 | 0.60 | 0.00 | 0.00 | 0.00 |
| 26 | 0.00 | 0.00 | 0.00 | 0.28 | 0.00 | 0.00 | 0.82 | 0.50 | 1.05 | 0.90 | 0.44 | 0.48 | 0.40 | 0.58 | 0.72 | 1.10 | 0.50 | 0.52 | 0.32 | 0.00 |
| 27 | 0.00 | 0.00 | 0.44 | 0.44 | 0.78 | 0.84 | 0.84 | 0.92 | 1.02 | 1.00 | 1.52 | 1.48 | 1.88 | 1.88 | 2.38 | 1.42 | 0.54 | 0.24 | 0.00 | 0.00 |
| 28 | 0.00 | 0.82 | 0.86 | 0.95 | 1.10 | 1.32 | 0.92 | 0.88 | 1.08 | 1.20 | 0.88 | 0.84 | 0.80 | 0.80 | 0.76 | 1.04 | 0.82 | 0.42 | 0.00 | 0.00 |
| N | 24 | 24 | 24 | 24 | 23 | 24 | 24 | 24 | 24 | 24 | 24 | 24 | 28 | 24 | 24 | 24 | 24 | 24 | 24 | 23 |
| Mean | 0.00 | 0.15 | 0.41 | 0.72 | 0.74 | 0.78 | 0.71 | 0.65 | 0.85 | 0.72 | 0.69 | 0.73 | 1.07 | 1.04 | 1.20 | 1.12 | 0.76 | 0.41 | 0.16 | 0.04 |
| Std. Dev | 0.00 | 0.28 | 0.32 | 0.41 | 0.35 | 0.38 | 0.28 | 0.22 | 0.29 | 0.33 | 0.30 | 0.35 | 0.52 | 0.40 | 0.43 | 0.44 | 0.28 | 0.24 | 0.18 | 0.10 |
| RSD % | 0.00 | 184.81 | 77.34 | 60.95 | 46.83 | 48.34 | 41.19 | 34.12 | 25.21 | 46.31 | 49.26 | 47.43 | 48.08 | 38.80 | 36.00 | 39.41 | 36.70 | 59.28 | 117.49 | 266.08 |

Note:
Concentrations were dose-adjusted to 24 mg.

TABLE 20
SUMMARY OF THE ADJUSTED PLASMA HYDROMORPHONE CONCENTRATION (NG/ML) AT EACH SAMPLING TIME BY SUBJECT

| | |
|---|---|
| SUBJECT | TREATMENT: Diluted 6 ng Fasted |

| NUMBER | 0 | 0.25 | 0.5 | 1 | 1.5 | 2 | 3 | 4 | 5 | 6 | 8 | 10 | 12 | 14 | 18 | 24 | 38 | 48 | 80 | 72 |
|---|---|---|---|---|---|---|---|---|---|---|---|---|---|---|---|---|---|---|---|---|
| 1 | 0.00 | 2.73 | 5.07 | 4.14 | 1.95 | 1.62 | 1.41 | 0.83 | 0.81 | 0.54 | 0.60 | 0.42 | 0.36 | 0.60 | 0.42 | 0.00 | 0.00 | 0.00 | 0.00 | 0.00 |
| 3 | 0.00 | 2.46 | 4.32 | 5.16 | 2.85 | 2.01 | 1.14 | 0.84 | 0.51 | 0.42 | 0.00 | 0.00 | 0.39 | 0.42 | 0.54 | 0.57 | 0.36 | 0.00 | 0.36 | 0.00 |
| 4 | 0.00 | 3.87 | 5.88 | 4.74 | 2.64 | 1.86 | 1.38 | 0.84 | 0.48 | 0.60 | 0.00 | 0.45 | 0.72 | 0.57 | 0.80 | 0.81 | 0.60 | 0.00 | 0.00 | 0.00 |
| 5 | 0.00 | 6.72 | 11.64 | 9.99 | 5.58 | 4.56 | 3.00 | 1.95 | 1.17 | 0.90 | 0.54 | 0.51 | 0.51 | 0.83 | 1.26 | 0.96 | 0.72 | 0.00 | 0.00 | 0.00 |
| 6 | 0.00 | 1.63 | 6.31 | 3.19 | 1.71 | 1.92 | 0.84 | 0.87 | 1.35 | 0.86 | . | . | 0.00 | 0.00 | 0.00 | 0.00 | 0.00 | 0.00 | 0.00 | 0.00 |
| 7 | 0.00 | 3.09 | 6.25 | 3.24 | 3.45 | 1.80 | 0.98 | 1.05 | 0.81 | 0.45 | 0.48 | 0.38 | 0.00 | 0.42 | 0.38 | 0.00 | 0.00 | 0.00 | 0.00 | 0.00 |
| 8 | 0.00 | 6.76 | 10.20 | 8.10 | 5.91 | 4.60 | 2.87 | 1.80 | 0.87 | 0.83 | 0.72 | 0.54 | 1.28 | 1.11 | 0.98 | 0.99 | 0.42 | 0.36 | 0.36 | 0.00 |
| 9 | 0.00 | 4.71 | 8.22 | 6.54 | 4.17 | 3.09 | 1.86 | 1.50 | 0.93 | 0.63 | 0.00 | 0.00 | 0.00 | 0.46 | 0.89 | 0.72 | 0.84 | 0.00 | 0.00 | 0.00 |
| 10 | 0.00 | 2.40 | 7.66 | 8.19 | 4.62 | 3.61 | 2.55 | 1.83 | 1.23 | 0.83 | 0.01 | 0.66 | 1.35 | 1.02 | 0.81 | 0.70 | 0.60 | 0.00 | 0.00 | 0.00 |
| 11 | 0.00 | 4.50 | 13.56 | 11.52 | 4.32 | 2.94 | 2.19 | 1.32 | 1.08 | 0.87 | 0.36 | 0.00 | 0.00 | 0.48 | 1.29 | 0.84 | 0.00 | 0.00 | 0.00 | 0.75 |
| 12 | 0.00 | 7.17 | 7.69 | 7.89 | 4.11 | 2.67 | 1.56 | 1.29 | 1.02 | 0.78 | 0.45 | 0.33 | 1.05 | 0.54 | 0.45 | 0.30 | 0.00 | 0.00 | 0.00 | 0.00 |
| 13 | 0.00 | 3.42 | 10.35 | 6.36 | 3.87 | 2.88 | 2.10 | 1.53 | 0.99 | 0.67 | 0.00 | 0.30 | 0.72 | 0.54 | . | 0.84 | 0.93 | 0.00 | 0.00 | 0.00 |
| 14 | 0.00 | 4.41 | 4.44 | 6.51 | 4.14 | 8.21 | 2.46 | 1.77 | 1.17 | 0.81 | 0.42 | 0.00 | 0.38 | 0.66 | 0.81 | 0.48 | 0.33 | 0.00 | 0.00 | 0.00 |
| 15 | 0.00 | 4.71 | 2.68 | 8.22 | 3.57 | 3.00 | 1.77 | 0.99 | 0.89 | 0.72 | 0.00 | 0.00 | 0.00 | 0.51 | 0.00 | 0.00 | 0.00 | 0.00 | 0.00 | 0.00 |
| 16 | 0.00 | 12.72 | 21.51 | 10.68 | 4.86 | 4.77 | 2.67 | 3.33 | 1.58 | 1.17 | 1.05 | 0.42 | 0.39 | 0.30 | 0.78 | 1.26 | 0.45 | 0.00 | 0.00 | 0.00 |
| 17 | 0.00 | 0.54 | 3.87 | 6.30 | 3.48 | 3.57 | 1.80 | 0.95 | 1.05 | 0.69 | 0.54 | 0.54 | 0.90 | 0.54 | 0.69 | 0.48 | 0.39 | 0.00 | 0.57 | 0.00 |
| 19 | 0.00 | 3.00 | 3.09 | 5.76 | 2.49 | 1.65 | 3.12 | 1.56 | 1.23 | 0.81 | 0.69 | 0.57 | 0.45 | 0.54 | 0.48 | 0.75 | 0.39 | 0.00 | 0.00 | 0.00 |
| 21 | 0.00 | 2.37 | 5.19 | 4.89 | 2.22 | 1.62 | 2.04 | 1.35 | 0.98 | 0.69 | 0.48 | 0.83 | 0.84 | 0.78 | 0.98 | 0.60 | 0.00 | 0.00 | 0.00 | 0.00 |
| 22 | 0.00 | 0.84 | 2.22 | 4.35 | 6.18 | 2.46 | 1.08 | 0.98 | 1.05 | 0.75 | 0.38 | 0.45 | 0.83 | 0.75 | 0.63 | 0.00 | 0.00 | 0.00 | 0.00 | 0.00 |
| 23 | 0.00 | 1.35 | 3.80 | 8.63 | 4.14 | 3.00 | 1.98 | 1.41 | 0.72 | 0.57 | 0.00 | 0.45 | 0.75 | 0.89 | 0.69 | 0.80 | 0.39 | 0.00 | 0.00 | 0.00 |
| 25 | 0.00 | 2.52 | 4.29 | 7.05 | 3.81 | 2.22 | 2.07 | 1.32 | 0.95 | 0.89 | 0.83 | 0.60 | 1.02 | 1.05 | 0.83 | 0.36 | 0.00 | 0.00 | 0.00 | 0.00 |
| 26 | 0.00 | 0.51 | 1.23 | 6.93 | 5.43 | 4.08 | 2.64 | 1.65 | 1.11 | 0.57 | 0.33 | 0.84 | 0.78 | 0.63 | 0.72 | 0.80 | 0.81 | 0.42 | 0.00 | 0.00 |
| 27 | 0.00 | 0.00 | 0.61 | 9.00 | 6.38 | 6.07 | 2.12 | 1.72 | 1.68 | 1.20 | 0.85 | 1.11 | 1.32 | 1.53 | 0.87 | 0.78 | 0.83 | 0.00 | 0.00 | 0.00 |
| 28 | 0.00 | 1.86 | 1.68 | 11.70 | 6.87 | 3.87 | 2.07 | 1.62 | 0.87 | 0.86 | 0.88 | 0.42 | 0.35 | 0.33 | 0.57 | 0.66 | 0.00 | 0.00 | 0.00 | 0.00 |
| N | 24 | 24 | 24 | 24 | 24 | 24 | 24 | 24 | 24 | 24 | 28 | 23 | 24 | 24 | 23 | 24 | 24 | 24 | 24 | 24 |
| Mean | 0.00 | 3.52 | 6.25 | 7.08 | 4.11 | 3.03 | 1.85 | 1.44 | 1.01 | 0.76 | 0.48 | 0.43 | 0.59 | 0.84 | 0.87 | 0.55 | 0.80 | 0.03 | 0.05 | 0.03 |
| Std. Dev | 0.00 | 2.77 | 4.66 | 2.47 | 1.42 | 1.07 | 0.61 | 0.54 | 0.28 | 0.21 | 0.80 | 0.30 | 0.43 | 0.31 | 0.32 | 0.86 | 0.20 | 0.11 | 0.15 | 0.15 |
| RSD % | 0.00 | 78.80 | 74.51 | 34.87 | 34.61 | 35.84 | 31.01 | 37.27 | 28.48 | 27.23 | 70.88 | 69.53 | 72.12 | 48.97 | 47.89 | 83.87 | 98.39 | 339.88 | 276.33 | 488.90 |

Note:
Concentrations were dose-adjusted to 24 mg.

### EXAMPLE 12

In Example 12, an open-label, 5-day, repeated-dose, two-treatment, randomized, cross-over, analytically blinded pharmacokinetic/pharmacodynamic comparision study of Hydromorphone HCL sustained release 12 mg capsules (HHCR 12 mg of Example 9) administered once daily and Hydromorphone HCL immediate release 3 mg tablets administered every 6 hours was conducted in normal, healthy, young male and female volunteers. The objective was to assess relative bioavailabilities (including gender effects) and to compare the pharmacokinetic / pharmacodynamic (PK/PD) profiles of HHCR 12 mg capsules administered once daily (“q24 h”) and HHIR 3 mg tablets administered every 6 hours (“q 6 h”) under apparent steady-state (multiple-dose) conditions. A total of 26 subjects (15 males and 11 females) were enrolled and completed the study. All were included in the both the PK/PD and safety analysis.

The hydromorphone hydrochloride controlled-release capsule (HHCR) tested in this example provides favorable release characteristics and plasma concentration-time profiles for once-daily administration. In Example 11, the results of the single-dose study showed that the 24 mg capsule was dose proportional to the 12 mg capsule based on primary pharmacokinetic metrics, and the pharmacokinetic profiles support once-daily dosing. The present study demonstrates the same results for the same formulation tested under steady-state conditions. Blood samples for determination of hydromorphone concentrations were obtained within 15 minutes before dosing (0 hour) on Days 2-5 of each study period, and at 0.25, 0.5, 1, 2, 3, 4, 6, 6.25, 6.5, 7, 8, 9, 10, 12, 12.25, 12.5, 13, 14, 15, 16, 18, 18.25, 18.5, 19, 20, 21, 22, and 24 hours (±2 minutes) after the 8 a.m. dose on Day 5 of each study period. Hydromorphone concentrations were determined using a validated APCI LS/MS/MS technique.

With respect to pharmacokinetics, measurements included: Area under the plasma concentration time course curve from dosing to 24h at steady-state [AUCss,(0-24)], maximum plasma concentration at steady-state [Css,max], minimum plasma concentration at steady-state [Css,min], minimum plasma concentration prior to steady-state [Ct,min], time to maximum plasma concentration at steady-state [tss,max], % fluctuation at steady-state, and time from initiation of therapy to steady-state [Tss].

Table 21 provides a summary of adjusted hydromorphone pharmacokinetic metrics (mean and standard deviation) for HHCR 12 mg and HHIR 3 mg.

TABLE 21

| | Arithmetic mean (SD) | |
|---|---|---|
| | HHCR 12 mg | HHIR 3 mg |
| Metric | q24 h | q6 h |
| AUCss,(0-24) (ng/mL · h) | | |
| Arithmetic Mean (SD) | 34.86 (10.15) | 34.40 (8.93) |
| Geometric Mean | 33.89 | 33.20 |
| Css,max (ng/mL) | | |
| Arithmetic Mean (SD) | 2.12 (0.64) | 2.89 (0.96) |
| Geometric Mean | 2.03 | 2.73 |
| Css,min (ng/mL) | | |
| Arithmetic Mean | 0.99 (0.35) | 0.70 (0.21) |
| Geometric Mean | 0.92 | 0.66 |
| Ct,min (ng/mL) | 1.26 (0.41) | 0.99 (0.23) |
| tss,max(h) | 8.44 (6.34) | 0.86 (0.52) |
| % Fluctuation | 125.80 (62.04) | 327.92 (124.32) |
| Tss(d) | 3 | 3 |
| Trough (ng/ml) | | |
| Arithmetic Mean (SD) | 1.26 (0.41) | 0.99 (0.23) |
| Geometric Mean | 1.18 | 0.96 |

SD = Standard deviation

The pharmacokinetic metrics used in the study are defined as follows:

AUCss,(0-24) (ng/mL•h)—The area under the plasma concentration-time course curve at steady-state from 0 to 24 hours (within one dosing interval).

Css,max (ng/mL)—The maximum observed plasma concentration at steady-state.

Css,min (ng/mL)—The minimum observed plasma concentration at steady-state (between 8 am on Day 5 and 8 a.m. on Day 6).

Ct,min (ng/mL)—The minimum plasma concentrations prior to steady-state (8 am on Days 2 - 5). tss,max (hr)—The time from dosing at steady-state to Css,max.

Fluctuation (%)—The difference between Css,max and Css,min expressed as a percentage of Css,min.

Tss (days)—The time from initiation of therapy to steady-state (the first day between Days 2 and 5 when the slope of Ct,min values no longer increases).

A summary of AUCss,(0-24) and Css,max and primary pharmacokinetic metrics (mean and standard deviation) and 90% confidence intervals around ratios (test/reference) of least squares means derived from logarithmic-transformed data are presented below in Table 22.

TABLE 22

| | Arithmetic mean (SD) | | | | |
|---|---|---|---|---|---|
| | HHCR | HHIR | | | |
| | 12 mg | 3 mg | LSM | | |
| Metric | q24 h | q6 h | Ratio (%)* | 90% CI^{b} | |
| AUCss,(0-24) | 34.86 | 34.40 | 101.36 | 96.31 | 106.40 |
| (ng/mL · h) | (10.15) | (8.93) | | | |
| Css,max | 2.12 (0.64) | 2.89 | 73.33 | 64.31 | 82.34 |
| (ng/mL) | | (0.96) | | | |

SD = Standard deviation.
^{a}Ratio (%) (test/reference) of least square means (ANOVA) derived from logarithmic-transformed data.
^{b}90% confidence interval (CI) around least squares means ratio.

The Ct,min data is presented in Table 23. Steady-state appeared to have been reached between Day 3 and 4 for both treatments.

TABLE 23
Summary of Mean Ct, min (ng/mL) (Mean. N = 26)

| | Study Day | | | | Overall |
|---|---|---|---|---|---|
| Treatment | 2 | 3 | 4 | 5 | Mean |
| HHCR | 0.69 | 0.99 | 1.26 | 1.26 | 1.05 |
| HHIR 0.55 | 0.77 | 0.89 | 0.99 | 0.80 | |

A summary of the time to maximum concentration (Tmax) by subject (HHCR 12 mg and HHIR 3 mg) is provided in Table 25. A summary of the maximum concentration (Cmax) by subject is provided in Table 26. A summary of the minimum concentration (Cmin) is provided in Table 27. A summary of the area under the curve (0-24 hours) by subject is provided in Table 28. A summary of the percent fluctuation in concentration (Cmin) by subject is provided in Table 29. A summary of the trough concentration values (day 5) by subject is provided in Table 30.

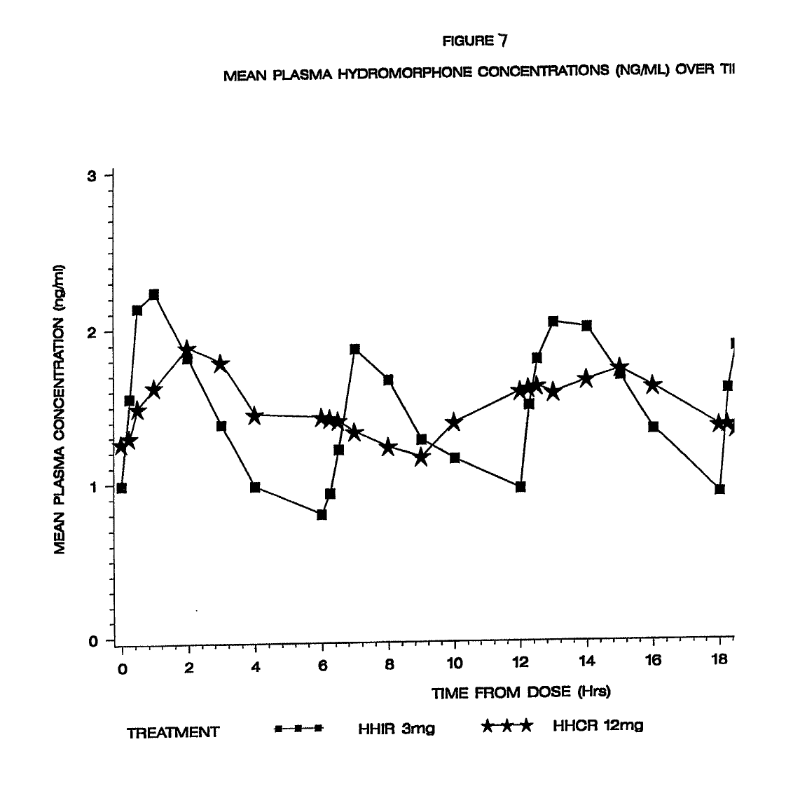

Mean plasma concentration-time course data for Example 12 are presented in FIG. 7. Each of the 4 daily administrations of HHIR 3 mg tablets produced a rapid initial increase in plasma drug concentration (tss,max by 1 hour) followed by a relatively rapid decline over the remaining 5 hours of the dosing interval. Each daily administration of HHCR capsules resulted in a relatively rapid rise to an initial early peak concentration, followed by a second broader peak with plateau concentrations maintained to 24 hours. A summary of the plasma hydromorphone concentration (ng/ml) values at each sampling time by subject is provided in Table 24.

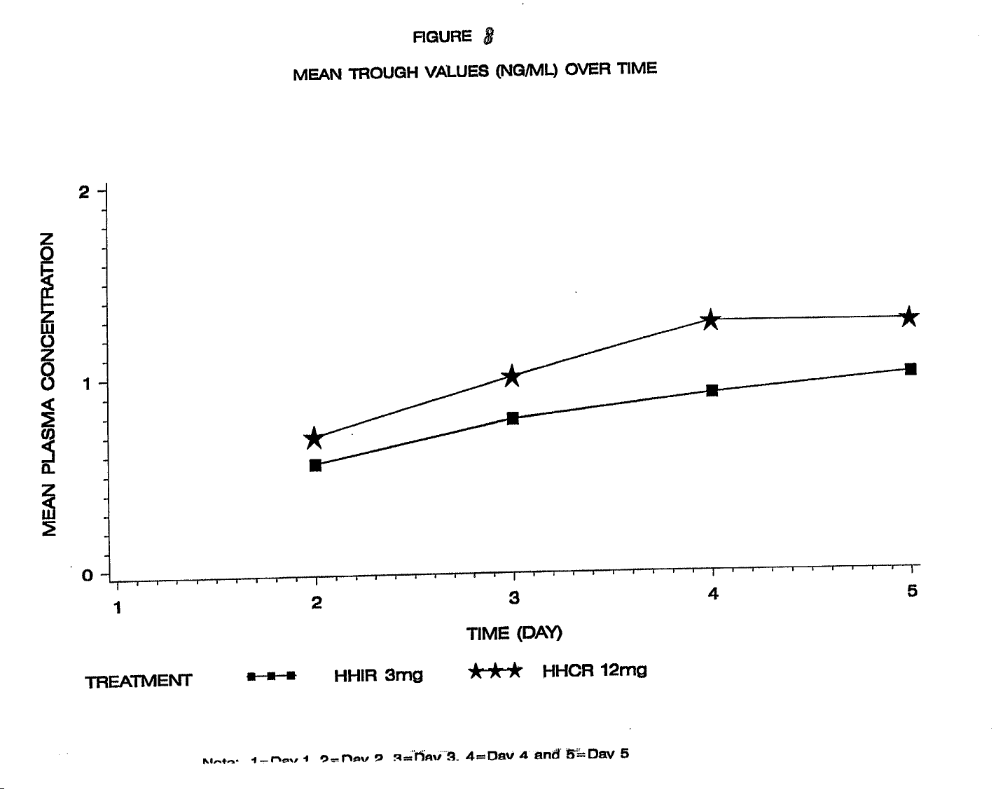

Comparisons of AUCss,(0-24) indicated that HHCR 12 mg q24h and HHIR 3 mg q6h are bioequivalent in terms of extent of absorption over a 24 hour period at steady-state. Mean AUCss,(0-24) values differed between treatments by<1% (CI=96 - 106). Comparison of Css,max values indicated that HHCR 12 mg capsules q24h had a 26% lower Css,max than HHIR 3 mg tablets q6h (CI=64 - 82), consistent with the sustained release characteristics of HHCR. The mean Css,min of HHCR 12 mg capsules q24h was higher than that of HHIR 3 mg tablets q6h (0.99 and 0.70 ng/mL, respectively), consistent with the controlled release characteristics of HHCR. The mean Ct,min of HHCR 12 mg capsules q24h was higher than that of HHIR 3 mg tablets q6h (1.26 and 0.99 ng/mL, respectively), consistent with the controlled release characteristics of HHCR capsules. The mean tss,max of HHCR 12 mg capsules q24h was considerably longer than that of HHIR 3 mg tablets (8.44 and 0.86 h, respectively). Considering the anticipated within-subject variability of tss,max, this observation was not of any significance. The fluctuation between Css,max and Css,min resulting from HHCR 12 mg capsules q24h was less than that from HHIR 3 mg tablets q6h (125.80 and 327.92 hours, respectively), consistent with the controlled release characteristics of HHCR capsules. The time to steady-state for most subjects was 3 days after the first dose of either treatment. Two subjects who received HHIR 3 mg tablets q6h and two subjects who received HHCR 12 mg q24h did not appear to reach steady-state on Day 3. All subjects had reached steady-state prior to the pharmacokinetic sampling period on Day 5. FIG. 8 provides the mean trough values (ng/ml) over time for HHIR and HHCR.

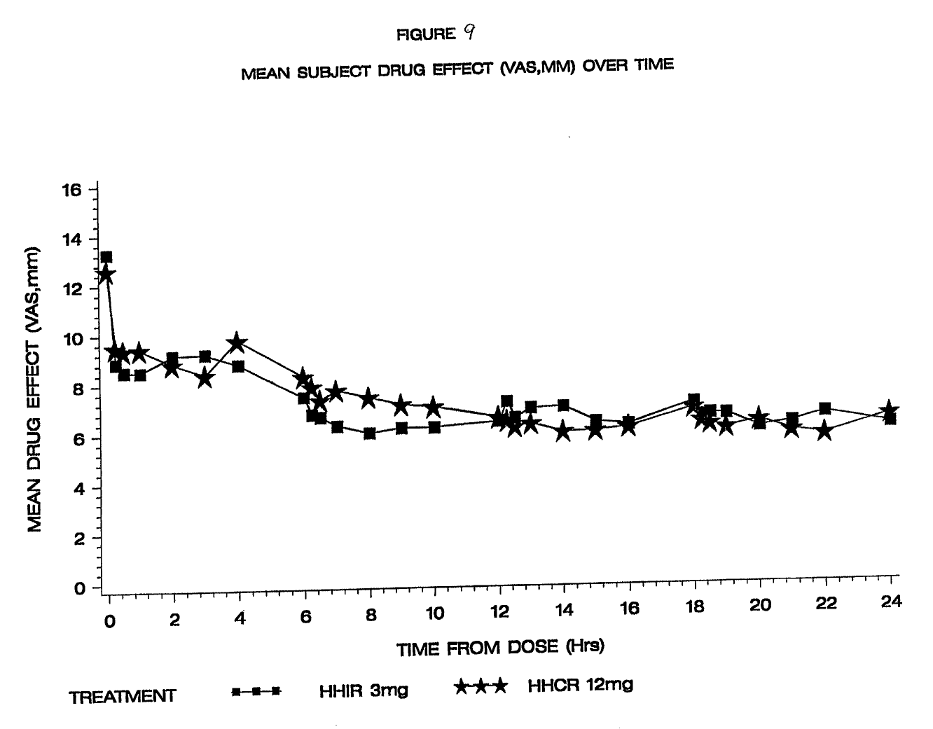

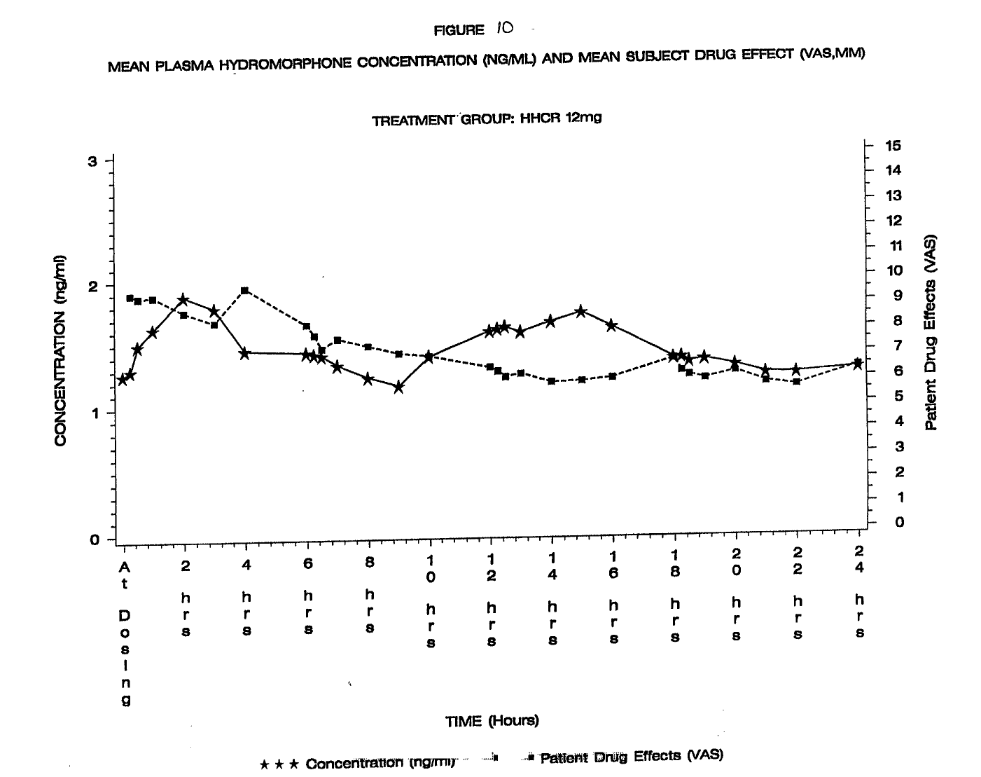

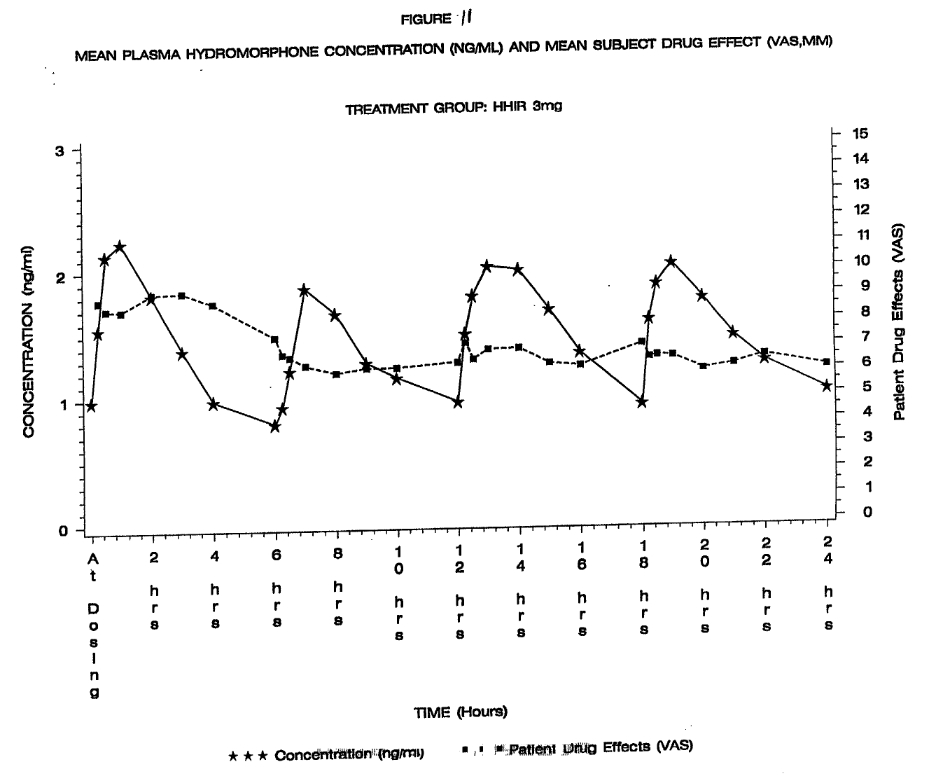

Relationships between the time course of plasma drug concentrations and pharmacodynamics (visual analog scale (VAS) “Drug Effects”) were assessed. The “drug effect” visual analog scale was chosen as the pharmacodynamic outcome because of its high correlation with plasma opioid concentrations relative to other subjective and objective assessments utilized in normal volunteer studies. The VAS “drug effect” measured just prior to blood sampling on Day 5 at baseline (within 30 minutes prior to dosing) and within 5 minutes prior to all scheduled times of blood draws. “Drug effect” consisted of one question, rated by the subject: “Do you feel any effects of the drug?” Just prior to blood sampling, the subject rated “drug effect” by placing a vertical mark along a 100 mm visual analog scale (VAS) anchored on one end by “not at all” and on the other end by “an awful lot”. With respect to pharmacokinetic-pharmacodynamic metrics, PDmax (maximum observed effect score) and PDauc (area under the effect curve from 0 to 24 hours) were estimated based on subject VAS “drug effect” scores. FIG. 9 provides the mean subject drug effect (VAS, mm) over time for HHIR and HHCR. FIG. 10 provides a graphical representation of the mean plasma hydromorphone concentration (ng/ml) and mean subject drug effect (VAS, mm) over time for HHCR. FIG. 11 provides a graphical representation of the mean plasma hydromorphone concentration (ng/ml) and mean subject drug effect (VAS, mm) over time for HHIR.

TABLE 24
SUMMARY OF THE PLASMA HYDROMORPHONE CONCENTRATION (MG/ML) VALUES AT EACH SAMPLING TIME BY SUBJECT

| SUBJECT | TREATMENT: HHCR 12 mg | | | | | | | | | | | | | | | | | | |
|---|---|---|---|---|---|---|---|---|---|---|---|---|---|---|---|---|---|---|---|
| NUMBER | −4 | −3 | −2 | −1 | 0 | 0.25 | 0.5 | 1 | 2 | 3 | 4 | 6 | 6.25 | 6.5 | 7 | 8 | 9 | 10 | 12 |
| 1 | 0.0 | 0.7 | 1.0 | 1.3 | 1.1 | 1.1 | 1.4 | 1.4 | 1.4 | 1.5 | 1.2 | 1.8 | 1.8 | 1.5 | 1.2 | 1.0 | 0.8 | 1.4 | 1.9 |
| 2 | 0.0 | 0.4 | 1.0 | 1.0 | 1.3 | 1.2 | 1.5 | 1.4 | 1.4 | 1.4 | 1.3 | 1.1 | 1.2 | 1.2 | 1.2 | 1.0 | 0.8 | 0.9 | 0.9 |
| 3 | 0.0 | 0.5 | 0.7 | 0.7 | 1.0 | 0.8 | 1.1 | 1.3 | 1.3 | 1.2 | 1.1 | 0.8 | 1.0 | 1.0 | 0.8 | 0.9 | 0.8 | 1.2 | 0.9 |
| 4 | 0.0 | 0.3 | 0.4 | 0.5 | 0.6 | 0.7 | 0.8 | 0.9 | 0.9 | 1.0 | 0.8 | 0.7 | 0.8 | 0.7 | 0.7 | 0.8 | 0.7 | 0.8 | 0.7 |
| 5 | 0.0 | 0.5 | 0.8 | 1.0 | 1.2 | 1.1 | 1.2 | 1.3 | 1.6 | 1.6 | 1.5 | 1.3 | 1.4 | 1.4 | 1.3 | 1.2 | 1.2 | 1.2 | 1.0 |
| 6 | 0.0 | 0.8 | 1.5 | 1.8 | 2.0 | 1.9 | 2.2 | 2.3 | 2.4 | 2.4 | 1.6 | 1.7 | 1.7 | 1.7 | 1.8 | 1.5 | 1.5 | 1.9 | 2.5 |
| 7 | 0.0 | 0.9 | 1.0 | 1.4 | 1.4 | 1.4 | 1.5 | 1.7 | 2.0 | 2.0 | 1.7 | 1.8 | 1.8 | 1.8 | 1.8 | 1.5 | 1.5 | 1.4 | 1.9 |
| 8 | 0.0 | 1.2 | 1.4 | 1.6 | 2.0 | 1.8 | 1.8 | 2.4 | 2.4 | . | 2.1 | 2.0 | 1.9 | 1.7 | 1.7 | 1.5 | 1.6 | 1.5 | 1.6 |
| 9 | 0.0 | 1.0 | 1.1 | 2.0 | 1.8 | 1.4 | 1.5 | 2.0 | 2.2 | 1.9 | 1.7 | 1.7 | 1.7 | 1.8 | 1.5 | 1.4 | 1.2 | 1.3 | 1.2 |
| 10 | 0.0 | 0.5 | 1.2 | 1.7 | 1.3 | 1.3 | 1.5 | 1.6 | 2.5 | 2.1 | 1.7 | 1.5 | 1.5 | 1.6 | 1.4 | 1.4 | 1.2 | 1.3 | 2.2 |
| 11 | 0.0 | 0.7 | 0.9 | 1.2 | 1.4 | 1.5 | 1.6 | 1.8 | 1.9 | 2.0 | 1.8 | 1.7 | 1.6 | 1.6 | 1.6 | 1.2 | 1.3 | 1.3 | 1.4 |
| 12 | 0.0 | 0.8 | 1.1 | 1.2 | 1.8 | 1.6 | 1.9 | 1.8 | 2.2 | 1.9 | 1.8 | 1.4 | 1.4 | 1.5 | 1.4 | 1.2 | 1.2 | 1.4 | 1.6 |
| 13 | 0.0 | 0.6 | 0.7 | 1.2 | 1.2 | 1.4 | 1.5 | 1.6 | . | 1.6 | 1.4 | 1.2 | 1.4 | 1.3 | 1.2 | 1.0 | 0.9 | 1.1 | 1.1 |
| 14 | 0.0 | 0.7 | 1.2 | 1.3 | 1.2 | 1.3 | 1.5 | 1.7 | 2.4 | 2.2 | 1.2 | 1.9 | 1.8 | 1.9 | 1.6 | 1.3 | 1.2 | 1.6 | 1.8 |
| 15 | 0.0 | 0.7 | 1.0 | 1.2 | 1.2 | 1.3 | 1.4 | 1.5 | 2.0 | 1.7 | 1.4 | 1.2 | 1.3 | 1.3 | 1.3 | 1.3 | 1.3 | 1.4 | 2.2 |
| 16 | 0.0 | 1.2 | 1.4 | 1.5 | 1.6 | 1.5 | 1.5 | 1.9 | 2.5 | 2.2 | 1.7 | 1.7 | 1.6 | 1.6 | 1.6 | 1.8 | 1.9 | 2.0 | 2.7 |
| 17 | 0.0 | 0.7 | 1.0 | 1.2 | 0.3 | 0.4 | 0.7 | 0.8 | 1.1 | 1.0 | 0.8 | 0.8 | 0.8 | 0.8 | 0.7 | 0.6 | 0.6 | 1.2 | 0.8 |
| 18 | 0.0 | 0.6 | 1.2 | 1.2 | 0.7 | 0.7 | 1.1 | 1.2 | 1.3 | 1.2 | 1.1 | 0.9 | 0.9 | 0.8 | 1.1 | 1.0 | 1.0 | 1.2 | 1.1 |
| 19 | 0.0 | 0.5 | 0.7 | 1.1 | 1.1 | 1.1 | 1.1 | 1.0 | 1.6 | 1.3 | 1.1 | 1.2 | 0.7 | 0.9 | 0.9 | 0.8 | 0.8 | 0.9 | 0.8 |
| 20 | 0.0 | 0.7 | 0.8 | 1.3 | 1.1 | 1.2 | 2.1 | 1.7 | 1.8 | 1.5 | 0.8 | 1.2 | 1.1 | 1.3 | 1.2 | 1.4 | 1.1 | 1.8 | 2.0 |
| 21 | 0.0 | 0.6 | 0.9 | 0.8 | 1.0 | 1.2 | 1.7 | 1.7 | 2.1 | 1.8 | 1.5 | 1.5 | 1.5 | 1.5 | 1.4 | 1.5 | 1.5 | 1.9 | 2.1 |
| 22 | 0.0 | 0.4 | 1.0 | 1.2 | 1.4 | 1.4 | 1.5 | 1.7 | 2.0 | 2.0 | 1.6 | 1.4 | 1.4 | 1.4 | 1.4 | 1.2 | 1.2 | 1.3 | 1.3 |
| 23 | 0.0 | 0.7 | 0.6 | 1.1 | 1.4 | 1.3 | 1.6 | 1.4 | 1.7 | 1.9 | 1.4 | 1.5 | 1.5 | 1.4 | 1.3 | 1.2 | 1.1 | 1.2 | 1.2 |
| 24 | 0.0 | 0.6 | 1.1 | 1.2 | 1.5 | 1.9 | 2.3 | 2.4 | 2.4 | 2.4 | 1.9 | 1.6 | 1.6 | 1.5 | 1.4 | 1.2 | 1.3 | 1.8 | 3.0 |
| 25 | 0.0 | 0.6 | 0.9 | 1.2 | 1.0 | 0.9 | 1.0 | 1.2 | 1.2 | 1.3 | 1.1 | 1.1 | 1.2 | 1.1 | 1.1 | 1.0 | 1.1 | 1.4 | 1.1 |
| 26 | 0.0 | 1.2 | 1.5 | 2.1 | 2.1 | 2.3 | 2.1 | 2.7 | 3.1 | 3.2 | 2.7 | 2.6 | 2.5 | 2.6 | 2.4 | 2.2 | 1.9 | 2.0 | 2.4 |
| N | 26 | 26 | 26 | 26 | 26 | 26 | 26 | 26 | 25 | 25 | 26 | 26 | 26 | 26 | 26 | 26 | 26 | 26 | 26 |
| Mean | 0.00 | 0.69 | 0.99 | 1.26 | 1.26 | 1.30 | 1.50 | 1.63 | 1.90 | 1.77 | 1.45 | 1.43 | 1.42 | 1.40 | 1.33 | 1.23 | 1.16 | 1.39 | 1.59 |
| Std. Dev | 0.00 | 0.25 | 0.26 | 0.36 | 0.41 | 0.41 | 0.42 | 0.47 | 0.53 | 0.50 | 0.42 | 0.42 | 0.40 | 0.40 | 0.35 | 0.34 | 0.33 | 0.33 | 0.65 |
| RSD % | 0.00 | 35.37 | 26.66 | 28.40 | 32.55 | 31.49 | 27.82 | 28.68 | 27.98 | 28.37 | 28.73 | 29.34 | 28.19 | 28.35 | 26.03 | 28.01 | 28.78 | 23.69 | 41.26 |

| SUBJECT | TREATMENT: HHCR 12 mg | | | | | | | | | | | | | |
|---|---|---|---|---|---|---|---|---|---|---|---|---|---|---|
| NUMBER | 12.25 | 12.5 | 13 | 14 | 15 | 16 | 18 | 18.25 | 18.5 | 19 | 20 | 21 | 22 | 24 |
| 1 | 2.0 | 1.9 | 1.7 | 1.7 | 1.6 | 1.8 | 1.2 | 1.3 | 1.2 | 1.1 | 1.3 | 1.1 | 1.2 | 1.3 |
| 2 | 1.0 | 0.9 | 0.9 | 1.1 | 1.0 | 0.9 | 0.7 | 0.7 | 0.7 | 0.8 | 0.9 | 1.0 | 0.9 | 0.9 |
| 3 | 0.9 | 0.8 | 0.7 | 0.6 | 0.6 | 0.6 | 0.4 | 0.5 | 0.5 | 0.5 | 0.5 | 0.5 | 0.4 | 0.4 |
| 4 | 0.8 | 0.8 | 0.7 | 0.8 | 1.0 | 0.8 | 0.6 | 0.6 | 0.6 | 0.6 | 0.6 | 0.6 | 0.6 | 0.6 |
| 5 | 1.2 | 1.2 | 1.2 | 1.2 | 1.3 | 1.4 | 1.1 | 1.0 | 1.0 | 0.9 | 0.9 | 1.0 | 0.8 | 1.2 |
| 6 | 2.5 | 2.5 | 2.6 | 2.9 | 2.6 | 2.5 | 2.1 | 2.4 | 2.2 | 2.4 | 2.1 | 2.2 | 1.9 | 1.8 |
| 7 | 1.8 | 1.7 | 1.4 | 1.5 | 1.4 | 1.4 | 1.3 | 1.2 | 1.2 | 1.1 | 1.2 | 1.3 | 1.4 | 1.6 |
| 8 | 1.8 | 1.9 | 1.8 | 2.1 | 2.5 | 2.3 | 1.7 | 1.7 | 1.7 | 1.6 | 1.7 | 1.4 | 1.6 | 1.8 |
| 9 | 1.2 | 1.2 | 1.3 | 1.4 | 1.8 | 1.5 | 1.2 | 1.2 | 1.2 | 1.2 | 1.2 | 1.4 | 1.8 | 2.2 |
| 10 | 2.2 | 2.2 | 1.9 | 2.0 | 1.6 | 2.2 | 2.1 | 2.0 | 1.9 | 1.8 | 1.3 | 1.5 | 1.4 | 1.3 |
| 11 | 1.4 | 1.5 | 1.6 | 1.5 | 1.6 | 1.3 | 1.3 | 1.5 | 1.7 | 2.2 | 1.7 | 1.6 | 1.6 | 1.5 |
| 12 | 1.4 | 1.4 | 1.3 | 1.3 | 1.6 | 1.4 | 1.2 | 1.0 | 0.9 | 1.1 | 1.1 | 1.3 | 1.0 | 1.3 |
| 13 | 0.9 | 1.2 | 1.0 | 1.0 | 1.4 | 1.2 | 1.1 | 1.0 | 1.1 | 1.1 | 1.0 | 1.0 | 0.9 | 1.1 |
| 14 | 1.9 | 1.7 | 1.5 | 1.7 | 1.9 | 2.0 | 1.4 | 1.4 | 1.5 | 1.5 | 1.5 | 1.2 | 1.2 | 1.0 |
| 15 | 1.9 | 2.3 | 2.4 | 1.7 | 1.9 | 1.8 | 1.7 | 1.6 | 1.6 | 1.5 | 1.3 | 1.2 | 1.3 | 1.3 |
| 16 | 2.9 | 2.8 | 1.4 | 2.6 | 2.4 | 2.9 | 1.9 | 1.8 | 1.8 | 1.6 | 1.7 | 1.7 | 1.7 | 1.4 |
| 17 | 0.9 | 0.9 | 0.8 | 0.8 | 0.8 | 0.6 | 0.6 | 0.5 | 0.5 | 0.5 | 0.8 | 0.6 | 0.6 | 0.8 |
| 18 | 1.1 | 1.0 | 1.2 | 1.2 | 1.2 | 1.1 | 1.2 | 1.3 | 1.2 | 1.0 | 1.1 | 1.1 | 0.9 | 0.9 |
| 19 | 0.7 | 0.8 | 0.9 | 1.2 | 1.7 | 1.1 | 1.4 | 1.5 | 1.5 | 1.4 | 1.2 | 1.1 | 1.2 | 0.8 |
| 20 | 1.9 | 2.1 | 1.9 | 2.7 | 2.2 | 2.2 | 1.6 | 1.5 | 1.4 | 1.4 | 1.1 | 0.9 | 0.9 | 1.2 |
| 21 | 2.0 | 2.1 | 2.0 | 2.2 | 2.2 | 2.0 | 1.7 | 1.8 | 1.6 | 1.7 | 1.6 | 1.4 | 1.4 | 1.4 |
| 22 | 1.5 | 1.3 | 2.8 | 1.4 | 2.0 | 1.6 | 1.6 | 1.6 | 1.5 | 1.6 | 1.5 | 1.4 | 1.3 | 1.2 |
| 23 | 1.2 | 1.3 | 1.2 | 1.2 | 1.3 | 1.1 | 1.8 | 1.4 | 1.4 | 1.3 | 1.4 | 1.2 | 1.2 | 1.6 |
| 24 | 2.9 | 3.1 | 3.0 | 2.7 | 2.2 | 2.0 | 1.4 | 1.4 | 1.2 | 1.3 | 1.2 | 1.1 | 1.1 | 1.0 |
| 25 | 1.2 | 1.1 | 1.1 | 1.1 | 1.5 | 1.7 | 1.5 | 1.5 | 1.5 | 1.6 | 1.6 | 1.3 | 1.1 | 0.9 |
| 26 | 2.4 | 2.5 | 2.6 | 3.5 | 3.6 | 2.6 | 2.1 | 2.2 | 2.1 | 2.4 | 2.2 | 2.4 | 2.4 | 2.6 |
| N | 26 | 26 | 26 | 26 | 26 | 26 | 26 | 26 | 26 | 26 | 26 | 26 | 26 | 26 |
| Mean | 1.60 | 1.62 | 1.57 | 1.66 | 1.73 | 1.61 | 1.38 | 1.38 | 1.33 | 1.35 | 1.30 | 1.23 | 1.23 | 1.26 |
| Std. Dev | 0.64 | 0.67 | 0.66 | 0.74 | 0.63 | 0.62 | 0.46 | 0.47 | 0.45 | 0.50 | 0.41 | 0.42 | 0.43 | 0.47 |
| RSD % | 40.15 | 41.23 | 42.03 | 44.42 | 36.73 | 38.81 | 33.53 | 34.44 | 34.15 | 38.95 | 31.91 | 34.03 | 35.05 | 37.57 |
| SUBJECT | TREATMENT: HHIR 3 mg | | | | | | | | | | | | | |
| NUMBER | 12.25 | 12.5 | 13 | 14 | 15 | 16 | 18 | 18.25 | 18.5 | 19 | 20 | 21 | 22 | 24 |
| 1 | 1.5 | 1.8 | 2.3 | 2.1 | 1.9 | 1.3 | 0.9 | 1.0 | 1.0 | 1.1 | 1.6 | 1.4 | 1.1 | 1.1 |
| 2 | 1.5 | 1.6 | 1.2 | 1.2 | 1.1 | 0.8 | 0.6 | 1.2 | 1.1 | 1.3 | 1.1 | 0.8 | 0.7 | 0.8 |
| 3 | 0.9 | 1.3 | 1.4 | 1.0 | 1.1 | 0.8 | 0.8 | 0.8 | 0.7 | 1.1 | 1.6 | 1.3 | 1.0 | 0.8 |
| 4 | 0.5 | 0.5 | 0.8 | 0.8 | 0.7 | 0.7 | 0.5 | 0.5 | 0.8 | 0.9 | 0.8 | 0.6 | 0.5 | 0.4 |
| 5 | 0.7 | 0.8 | 1.3 | 1.6 | 1.6 | 1.4 | 1.2 | 1.5 | 1.4 | 1.7 | 1.7 | 1.6 | 1.3 | 1.3 |
| 6 | 0.9 | 1.0 | 1.7 | 2.6 | 2.4 | 2.0 | 1.6 | 1.7 | 1.7 | 2.7 | 2.4 | 1.9 | 1.6 | 1.3 |
| 7 | 0.8 | 1.1 | 1.6 | 2.2 | 1.9 | 1.2 | 0.8 | 2.4 | 2.3 | 1.8 | 1.8 | 1.3 | 1.1 | 1.0 |
| 8 | 4.9 | 4.7 | 3.7 | 2.6 | 1.6 | 1.1 | 0.8 | 2.3 | 3.1 | 3.0 | 2.7 | 1.9 | 1.5 | 1.2 |
| 9 | 1.0 | 1.9 | 2.3 | 2.2 | 1.7 | 1.4 | 1.1 | 2.0 | 2.1 | 1.9 | 1.4 | 1.3 | 1.2 | 1.2 |
| 10 | 1.5 | 1.8 | 2.9 | 3.0 | 2.6 | 1.8 | 1.4 | 2.4 | 2.3 | 3.0 | 2.0 | 1.9 | 1.5 | 1.0 |
| 11 | 1.5 | 1.8 | 2.0 | 2.4 | 2.3 | 1.9 | 1.1 | 3.5 | 2.6 | 2.0 | 1.9 | 1.4 | 1.4 | 1.2 |
| 12 | 0.6 | 1.5 | 1.5 | 1.4 | 1.0 | 0.9 | 0.6 | 0.7 | 0.7 | 1.5 | 0.8 | 0.8 | 0.7 | 1.1 |
| 13 | 0.6 | 0.7 | 0.8 | 1.6 | 1.7 | 1.2 | 0.8 | 1.0 | 1.2 | 1.3 | 1.2 | 1.0 | 0.8 | 0.9 |
| 14 | 0.7 | 0.6 | 1.0 | 2.3 | 1.8 | 1.2 | 0.7 | 1.2 | 2.1 | 2.0 | 1.1 | 1.1 | 1.0 | 0.8 |
| 15 | 1.4 | 1.7 | 3.1 | 2.9 | 2.3 | 1.6 | 1.0 | 1.1 | 2.3 | 2.3 | 2.1 | 1.7 | 1.4 | 1.3 |
| 16 | 1.2 | 2.4 | 2.5 | 2.1 | 1.6 | 1.4 | 1.0 | 2.5 | 2.4 | 3.7 | 2.7 | 2.7 | 2.6 | 2.0 |
| 17 | 0.8 | 1.7 | 1.5 | 1.2 | 0.7 | 0.6 | 0.5 | 1.4 | 1.6 | 1.8 | 1.4 | 1.2 | 1.0 | 0.6 |
| 18 | 1.5 | 1.4 | 1.8 | 2.1 | 1.9 | 1.4 | 1.2 | 3.6 | 2.5 | 2.0 | 1.4 | 1.3 | 1.1 | 1.0 |
| 19 | 1.0 | 1.5 | 2.3 | 1.7 | 1.5 | 1.3 | 0.9 | 1.0 | 2.5 | 2.4 | 2.1 | 2.0 | 1.7 | 0.8 |
| 20 | 4.4 | 4.0 | 3.4 | 2.8 | 1.5 | 1.2 | 0.8 | 1.7 | 3.9 | 2.7 | 2.3 | 1.7 | 1.5 | 0.9 |
| 21 | 1.2 | 1.1 | 1.2 | 1.5 | 1.7 | 2.1 | 1.3 | 1.5 | 2.5 | 2.3 | 2.1 | 1.5 | 1.3 | 0.8 |
| 22 | 2.2 | 3.2 | 3.0 | 2.5 | 2.0 | 1.6 | 0.9 | 2.5 | 2.8 | 2.5 | 1.7 | 1.4 | 1.2 | 1.0 |
| 23 | 2.2 | 2.0 | 2.2 | 1.8 | 1.6 | 1.3 | 0.9 | 0.8 | 0.7 | 1.5 | 1.6 | 1.4 | 1.0 | 0.7 |
| 24 | 1.9 | 2.4 | 2.7 | 2.1 | 2.0 | 1.5 | 1.0 | 1.5 | 1.5 | 2.2 | 2.1 | 1.7 | 1.6 | 1.2 |
| 25 | 0.7 | 0.9 | 1.1 | 1.3 | 1.2 | 0.9 | 0.6 | 0.8 | 0.7 | 1.5 | 1.6 | 1.4 | 1.4 | 1.0 |
| 26 | 2.6 | 3.3 | 3.4 | 2.9 | 2.7 | 1.9 | 1.2 | 1.3 | 2.2 | 2.7 | 2.7 | 2.0 | 1.7 | 1.1 |
| N | 26 | 26 | 26 | 26 | 26 | 26 | 26 | 26 | 26 | 28 | 26 | 26 | 26 | 26 |
| Mean | 1.49 | 1.79 | 2.02 | 1.99 | 1.68 | 1.34 | 0.92 | 1.59 | 1.87 | 2.03 | 1.75 | 1.46 | 1.26 | 1.02 |
| Std. Dev | 1.09 | 1.04 | 0.85 | 0.61 | 0.52 | 0.41 | 0.28 | 0.83 | 0.84 | 0.68 | 0.54 | 0.46 | 0.42 | 0.80 |
| RSD % | 72.76 | 58.00 | 42.23 | 30.78 | 31.24 | 30.85 | 30.68 | 52.38 | 45.21 | 33.52 | 30.51 | 31.25 | 33.44 | 29.05 |

| SUBJECT | TREATMENT: HHIR 3 mg | | | | | | | | | | | | | | | | | | |
|---|---|---|---|---|---|---|---|---|---|---|---|---|---|---|---|---|---|---|---|
| NUMBER | −4 | −3 | −2 | −1 | 0 | 0.25 | 0.5 | 1 | 2 | 3 | 4 | 6 | 6.25 | 6.5 | 7 | 8 | 9 | 10 | 12 |
| 1 | 0.0 | 0.5 | 0.7 | 1.0 | 1.0 | 1.2 | 2.2 | 2.4 | 1.7 | 1.3 | 0.8 | 1.0 | 1.4 | 2.2 | 2.1 | 1.4 | 1.0 | 0.8 | 1.1 |
| 2 | 0.0 | 0.6 | 0.6 | 0.8 | 0.9 | 0.7 | 0.8 | 1.4 | 1.2 | 1.0 | 0.7 | 0.4 | 0.3 | 0.4 | 0.7 | 0.7 | 0.8 | 0.9 | 0.8 |
| 3 | 0.0 | 0.6 | 0.5 | 0.5 | 0.6 | 0.9 | 1.8 | 1.5 | 1.2 | 0.9 | 0.6 | 0.5 | 0.4 | 0.4 | 0.6 | 0.8 | 0.8 | 1.0 | 0.5 |
| 4 | 0.0 | 0.4 | 0.4 | 0.4 | 0.5 | 0.6 | 0.9 | 1.1 | 1.1 | 0.7 | 0.6 | 0.4 | 0.6 | 0.6 | 1.2 | 1.0 | 0.6 | 0.6 | 0.5 |
| 5 | 0.0 | 0.5 | 0.6 | 0.7 | 0.9 | 0.9 | 1.4 | 1.6 | 1.3 | 1.2 | 0.9 | 0.6 | 0.7 | 0.7 | 1.9 | 1.7 | 1.2 | 1.1 | 0.7 |
| 6 | 0.0 | 0.7 | 1.2 | 1.4 | 1.4 | 2.5 | 2.8 | 2.3 | 2.4 | 1.8 | 1.3 | 1.0 | 1.1 | 1.2 | 3.7 | 2.0 | 1.4 | 1.1 | 1.0 |
| 7 | 0.0 | 0.7 | 1.0 | 1.0 | 1.1 | 1.4 | 2.1 | 2.3 | 2.2 | 1.5 | 1.0 | 1.0 | 0.9 | 0.8 | 1.0 | 2.1 | 1.3 | 1.1 | 0.8 |
| 8 | 0.0 | 0.8 | 1.2 | 1.4 | 1.2 | 2.9 | 2.9 | 2.6 | 2.3 | 1.7 | 1.0 | 0.9 | 0.9 | 1.1 | 2.3 | 2.1 | 1.7 | 1.5 | 1.5 |
| 9 | 0.0 | 0.5 | 0.8 | 1.0 | 1.1 | 1.1 | 1.6 | 1.9 | 2.4 | 2.0 | 1.4 | 1.1 | 1.3 | 1.4 | 1.7 | 1.9 | 1.7 | 1.3 | 1.0 |
| 10 | 0.0 | 0.5 | 0.7 | 0.8 | 0.9 | 1.1 | 1.9 | 2.2 | 2.3 | 1.3 | 0.9 | 0.7 | 0.8 | 1.2 | 1.8 | 2.2 | 1.4 | 1.7 | 1.4 |
| 11 | 0.0 | 0.7 | 0.9 | 1.2 | 1.0 | 1.3 | 2.2 | 2.5 | 2.0 | 1.4 | 1.1 | 0.8 | 0.8 | 0.8 | 1.6 | 1.5 | 1.2 | 1.1 | 1.3 |
| 12 | 0.0 | 0.6 | 0.7 | 0.9 | 1.0 | 1.0 | 2.1 | 2.4 | 1.7 | 1.3 | 1.0 | 0.9 | 1.4 | 1.9 | 1.6 | 1.4 | 0.8 | 0.8 | 0.6 |
| 13 | 0.0 | 0.4 | 0.7 | 0.7 | 0.8 | 0.9 | 1.8 | 1.8 | 1.3 | 1.0 | 0.9 | 0.8 | 0.7 | 1.0 | 1.4 | 1.5 | 1.1 | 0.9 | 0.6 |
| 14 | 0.0 | 0.5 | 0.6 | 0.5 | 0.8 | 1.7 | 2.1 | 1.8 | 1.4 | 0.9 | 0.7 | 0.5 | 0.6 | 1.6 | 2.1 | 1.3 | 0.7 | 0.7 | 0.7 |
| 15 | 0.0 | 0.6 | 1.0 | 1.1 | 1.1 | 2.3 | 3.5 | 3.0 | 2.6 | 1.6 | 1.1 | 0.9 | 1.1 | 2.0 | 3.6 | 2.2 | 1.6 | 1.3 | 1.0 |
| 16 | 0.0 | 0.4 | 0.9 | 0.7 | 1.1 | 1.3 | 2.5 | 3.7 | 3.1 | 1.9 | 1.3 | 1.0 | 1.1 | 0.8 | 1.9 | 2.5 | 1.5 | 1.2 | 0.7 |
| 17 | 0.0 | 0.7 | 0.9 | 0.4 | 1.1 | 1.2 | 1.4 | 2.5 | 2.1 | 2.1 | 1.4 | 0.9 | 1.7 | 2.4 | 2.1 | 1.4 | 1.0 | 0.7 | 0.4 |
| 18 | 0.0 | 0.4 | 0.6 | 0.8 | 1.0 | 4.1 | 2.8 | 2.2 | 1.4 | 1.3 | 1.1 | 0.8 | 1.2 | 2.2 | 1.6 | 1.2 | 1.1 | 1.1 | 1.1 |
| 19 | 0.0 | 0.5 | 0.8 | 1.1 | 0.9 | 1.2 | 1.7 | 1.7 | 1.4 | 0.9 | 0.7 | 0.8 | 0.7 | 1.0 | 1.9 | 1.3 | 0.9 | 0.7 | 0.7 |
| 20 | 0.0 | 0.5 | 0.8 | 1.1 | 0.9 | 1.4 | 2.4 | 2.8 | 1.5 | 1.2 | 0.8 | 0.8 | 0.7 | 0.9 | 2.7 | 1.7 | 1.3 | 0.8 | 1.0 |
| 21 | 0.0 | 0.4 | 0.6 | 1.1 | 1.0 | 1.8 | 3.1 | 2.7 | 1.8 | 1.2 | 0.8 | 0.7 | 0.8 | 0.9 | 3.5 | 2.3 | 1.5 | 1.5 | 1.1 |
| 22 | 0.0 | 0.5 | 0.7 | 1.1 | 1.1 | 1.1 | 1.4 | 1.7 | 2.2 | 1.9 | 1.3 | 1.0 | 0.9 | 0.9 | 1.6 | 1.5 | 1.8 | 1.4 | 1.2 |
| 23 | 0.0 | . | 0.5 | 0.6 | 0.8 | 0.9 | 3.6 | 2.2 | 1.5 | 1.2 | 0.9 | 0.6 | 0.7 | 0.6 | 1.1 | 1.5 | 1.0 | 0.9 | 0.7 |
| 24 | 0.0 | 0.5 | 0.7 | 0.9 | 1.1 | 3.0 | 3.5 | 2.6 | 1.9 | 1.4 | 1.1 | 1.2 | 1.3 | 2.6 | 2.2 | 2.0 | 1.8 | 1.8 | 1.5 |
| 25 | 0.0 | 0.7 | 0.7 | 0.6 | 1.0 | 0.9 | 1.5 | 1.8 | 1.3 | 1.1 | 0.8 | 0.7 | 0.7 | 0.6 | 0.6 | 1.0 | 1.1 | 0.9 | 0.5 |
| 26 | 0.0 | 0.6 | 1.1 | 1.3 | 1.7 | 3.2 | 1.7 | 3.5 | 2.4 | 2.2 | 1.4 | 1.1 | 1.2 | 1.5 | 2.0 | 3.0 | 2.8 | 2.9 | 2.8 |
| N | 26 | 25 | 26 | 26 | 26 | 26 | 26 | 26 | 26 | 26 | 26 | 26 | 26 | 26 | 26 | 26 | 26 | 26 | 26 |
| Mean | 0.00 | 0.55 | 0.77 | 0.89 | 0.99 | 1.56 | 2.14 | 2.24 | 1.82 | 1.38 | 0.98 | 0.79 | 0.93 | 1.21 | 1.86 | 1.66 | 1.27 | 1.16 | 0.96 |
| Std. Dev | 0.00 | 0.12 | 0.21 | 0.28 | 0.23 | 0.90 | 0.76 | 0.63 | 0.53 | 0.40 | 0.26 | 0.23 | 0.33 | 0.64 | 0.83 | 0.54 | 0.47 | 0.48 | 0.49 |
| RSD % | 0.00 | 21.64 | 26.98 | 31.80 | 23.23 | 57.81 | 35.62 | 28.09 | 28.86 | 26.85 | 26.16 | 28.41 | 35.98 | 52.53 | 44.38 | 32.41 | 36.93 | 42.03 | 51.31 |

TABLE 25
SUMMARY OF THE TIME TO MAXIMUM CONCENTRATION
(TMAX) BY SUBJECT
HHCR 12 MG AND HHIR 3 MG

| | Tmax (hrs) | | |
|---|---|---|---|
| | | | Ratio (%) |
| Subject | Treatment | | HHCR 12 mg/ |
| Number | HHCR 12 mg | HHIR 3 mg | HHIR 3 mg |
| 1 | 12.25 | 1.00 | 1225.00 |
| 2 | 0.50 | 0.50 | 100.00 |
| 3 | 1.00 | 0.50 | 200.00 |
| 4 | 3.00 | 1.00 | 300.00 |
| 5 | 3.00 | 1.00 | 300.00 |
| 6 | 14.00 | 1.00 | 1400.00 |
| 7 | 2.00 | 0.25 | 800.00 |
| 8 | 15.00 | 0.25 | 6000.00 |
| 9 | 2.00 | 2.00 | 100.00 |
| 10 | 2.00 | 2.00 | 100.00 |
| 11 | 19.00 | 0.25 | 7600.00 |
| 12 | 2.00 | 1.00 | 200.00 |
| 13 | 1.00 | 1.00 | 100.00 |
| 14 | 2.00 | 2.00 | 100.00 |
| 15 | 13.00 | 1.00 | 1300.00 |
| 16 | 12.25 | 1.00 | 1225.00 |
| 17 | 10.00 | 1.00 | 1000.00 |
| 18 | 2.00 | 0.25 | 800.00 |
| 19 | 15.00 | 0.50 | 3000.00 |
| 20 | 14.00 | 0.25 | 5600.00 |
| 21 | 15.00 | 1.00 | 1500.00 |
| 22 | 13.00 | 0.50 | 2600.00 |
| 23 | 3.00 | 0.50 | 600.00 |
| 24 | 12.50 | 0.50 | 2500.00 |

*Value in parenthesis is the ratio of the treatment means.

TABLE 26
SUMMARY OF THE MAXIMUM CONCENTRATION
(CMAX) BY SUBJECT
HHCR 12 MG AND HHIR 3 MG

| | Cmax (ng/ml) | | |
|---|---|---|---|
| | | | Ratio (%) |
| Subject | Treatment | | HHCR 12 mg/ |
| Number | HHCR 12 mg | HHIR 3 mg | HHIR 3 mg |
| 1 | 2.00 | 2.38 | 84.03 |
| 2 | 1.51 | 1.59 | 94.97 |
| 3 | 1.30 | 1.76 | 73.86 |
| 4 | 1.02 | 1.16 | 87.93 |
| 5 | 1.58 | 1.90 | 83.16 |
| 6 | 2.89 | 3.70 | 78.11 |
| 7 | 2.03 | 2.39 | 84.94 |
| 8 | 2.46 | 4.91 | 50.10 |
| 9 | 2.18 | 2.40 | 90.83 |
| 10 | 2.48 | 2.98 | 83.22 |
| 11 | 2.16 | 3.53 | 61.19 |
| 12 | 2.19 | 2.43 | 90.12 |
| 13 | 1.61 | 1.83 | 87.98 |
| 14 | 2.43 | 2.30 | 105.65 |
| 15 | 2.38 | 3.55 | 67.04 |
| 16 | 2.92 | 3.72 | 78.49 |
| 17 | 1.17 | 2.52 | 46.43 |
| 18 | 1.32 | 4.12 | 32.04 |
| 19 | 1.67 | 2.45 | 68.16 |
| 20 | 2.71 | 4.44 | 61.04 |
| 21 | 2.23 | 3.53 | 63.17 |
| 22 | 2.77 | 3.23 | 85.76 |
| 23 | 1.86 | 3.62 | 51.38 |
| 24 | 3.06 | 3.47 | 88.18 |

*Value in parenthesis is the ratio of the treatment means.

TABLE 27
SUMMARY OF THE MINIMUM CONCENTRATION
(CMIN) BY SUBJECT
HHCR 12 MG AND HHIR 3 MG

| | Cmin (ng/ml) | | |
|---|---|---|---|
| | | | Ratio (%) |
| Subject | Treatment | | HHCR 12 mg/ |
| Number | HHCR 12 mg | HHIR 3 mg | HHIR 3 mg |
| 1 | 0.84 | 0.80 | 105.12 |
| 2 | 0.68 | 0.31 | 219.61 |
| 3 | 0.37 | 0.41 | 90.44 |
| 4 | 0.56 | 0.42 | 135.01 |
| 5 | 0.82 | 0.64 | 129.45 |
| 6 | 1.49 | 0.95 | 157.01 |
| 7 | 1.11 | 0.77 | 143.97 |
| 8 | 1.42 | 0.84 | 169.25 |
| 9 | 1.15 | 0.98 | 117.47 |
| 10 | 1.21 | 0.72 | 168.99 |
| 11 | 1.19 | 0.76 | 157.62 |
| 12 | 0.94 | 0.60 | 157.10 |
| 13 | 0.88 | 0.59 | 150.34 |
| 14 | 0.99 | 0.49 | 204.11 |
| 15 | 1.15 | 0.90 | 128.06 |
| 16 | 1.40 | 0.72 | 193.64 |
| 17 | 0.26 | 0.41 | 65.02 |
| 18 | 0.68 | 0.82 | 82.99 |
| 19 | 0.70 | 0.60 | 116.45 |
| 20 | 0.92 | 0.58 | 156.68 |
| 21 | 0.99 | 0.70 | 141.39 |
| 22 | 1.19 | 0.93 | 128.37 |
| 23 | 1.07 | 0.59 | 181.36 |
| 24 | 1.01 | 1.04 | 97.12 |

TABLE 28
SUMMARY OF THE AREA UNDER THE CURVE
(0-24 HRS) BY SUBJECT
HHCR 12 MG AND HHIR 3 MG

| | AUC (0-24 hrs) | | |
|---|---|---|---|
| | | | Ratio (%) |
| Subject | Treatment | | HHCR 12 mg/ |
| Number | HHCR 12 mg | HHIR 3 mg | HHIR 3 mg |
| 1 | 33.35 | 32.79 | 101.70 |
| 2 | 24.38 | 21.18 | 115.14 |
| 3 | 18.87 | 22.00 | 85.75 |
| 4 | 17.54 | 16.48 | 106.46 |
| 5 | 28.81 | 30.39 | 94.79 |
| 6 | 50.62 | 42.99 | 117.75 |
| 7 | 36.99 | 33.41 | 110.70 |
| 8 | 44.73 | 45.07 | 99.24 |
| 9 | 36.77 | 37.25 | 98.69 |
| 10 | 40.72 | 42.21 | 96.47 |
| 11 | 37.51 | 37.70 | 99.49 |
| 12 | 33.54 | 26.73 | 125.45 |
| 13 | 28.40 | 25.85 | 109.85 |
| 14 | 37.86 | 27.19 | 139.23 |
| 15 | 37.11 | 43.86 | 84.60 |
| 16 | 47.52 | 46.48 | 102.35 |
| 17 | 18.40 | 29.17 | 63.08 |
| 18 | 25.86 | 35.08 | 73.71 |
| 19 | 26.63 | 31.76 | 83.86 |
| 20 | 36.37 | 38.86 | 93.60 |
| 21 | 41.40 | 37.99 | 108.99 |
| 22 | 36.79 | 39.32 | 93.58 |
| 23 | 31.94 | 29.68 | 107.62 |
| 24 | 43.68 | 42.49 | 102.82 |

*Value in parenthesis is the ratio of the treatment means.

TABLE 29
SUMMARY OF THE PERCENT FLUCTUATION IN
CONCENTRATION (CMIN) BY SUBJECT
HHCR 12 MG AND HHIR 3 MG

| | % Fluctuation | | |
|---|---|---|---|
| | | | Ratio (%) |
| Subject | Treatment | | HHCR 12 mg/ |
| Number | HHCR 12 mg | HHIR 3 mg | HHIR 3 mg |
| 1 | 137.53 | 197.13 | 69.77 |
| 2 | 121.08 | 411.25 | 29.44 |
| 3 | 252.30 | 331.37 | 76.14 |
| 4 | 81.17 | 178.18 | 45.56 |
| 5 | 92.21 | 199.21 | 40.29 |
| 6 | 93.96 | 289.88 | 32.41 |
| 7 | 82.88 | 209.99 | 39.47 |
| 8 | 73.24 | 485.22 | 15.09 |
| 9 | 89.57 | 145.15 | 61.71 |
| 10 | 104.96 | 316.20 | 33.19 |
| 11 | 81.51 | 367.55 | 22.18 |
| 12 | 132.73 | 305.68 | 48.42 |
| 13 | 82.13 | 211.22 | 38.88 |
| 14 | 144.47 | 372.28 | 38.81 |
| 15 | 106.96 | 295.32 | 38.22 |
| 16 | 108.57 | 414.52 | 26.19 |
| 17 | 348.18 | 520.69 | 65.91 |
| 18 | 94.69 | 404.28 | 23.42 |
| 19 | 138.23 | 306.98 | 45.03 |
| 20 | 196.17 | 660.27 | 29.71 |
| 21 | 124.35 | 402.13 | 30.92 |
| 22 | 132.77 | 248.44 | 53.44 |
| 23 | 73.83 | 513.56 | 14.38 |
| 24 | 202.97 | 233.65 | 86.87 |

*Value in parenthesis is the ratio of the treatment means.

TABLE 30
SUMMARY OF TROUGH CONCENTRATION VALUES
(DAY 5) BY SUBJECT
HHCR 12 MG AND HHIR 3 MG

| | Trough (ng/ml) | | |
|---|---|---|---|
| | | | Ratio (%) |
| Subject | Treatment | | HHCR 12mg/ |
| Number | HHCR 12 mg | HHIR 3 mg | HHIR 3 mg |
| 1 | 1.12 | 1.01 | 110.89 |
| 2 | 1.26 | 0.89 | 141.41 |
| 3 | 0.96 | 0.62 | 155.43 |
| 4 | 0.63 | 0.48 | 130.17 |
| 5 | 1.21 | 0.92 | 132.24 |
| 6 | 1.99 | 1.42 | 140.14 |
| 7 | 1.36 | 1.12 | 121.43 |
| 8 | 1.99 | 1.15 | 173.04 |
| 9 | 1.60 | 1.08 | 148.15 |
| 10 | 1.28 | 0.92 | 139.13 |
| 11 | 1.41 | 1.00 | 141.00 |
| 12 | 1.55 | 0.99 | 156.41 |
| 13 | 1.17 | 0.76 | 154.15 |
| 14 | 1.20 | 0.78 | 154.64 |
| 15 | 1.19 | 1.09 | 109.17 |
| 16 | 1.55 | 1.05 | 147.62 |
| 17 | 0.26 | 1.09 | 24.22 |
| 18 | 0.68 | 0.96 | 70.33 |
| 19 | 1.07 | 0.85 | 125.44 |
| 20 | 1.14 | 0.95 | 120.63 |
| 21 | 0.99 | 0.97 | 102.69 |
| 22 | 1.35 | 1.05 | 128.57 |
| 23 | 1.35 | 0.83 | 163.44 |
| 24 | 1.47 | 1.06 | 138.68 |

*Value in parenthesis is the ratio of the treatment means.

The examples provided above are not meant to be exclusive. Many other variations of the present invention would be obvious to those skilled in the art, and are contemplated to be within the scope of the appended claims.

What is claimed is:

1. A solid sustained release once-a-day oral dosage form comprising hydromorphone or a pharmaceutically acceptable salt thereof together with a sustained release carrier, the dosage providing a relatively rapid rise in plasma concentration to a first initial early peak concentration (Cmax #1) in about 0.3 to about 4 hours after oral administration of the dosage form, followed by a second peak concentration (Cmax #2) which occurs in about 10 to about 19 hours after oral administration of the dosage form, said dosage form providing effective treatment of pain for about 24 hours or more after administration to a human patient.

2. The dosage form of claim 1, wherein said time to first peak plasma concentration (Tmax #1) of the hydromorphone occurs in about 1 to about 3 hours after oral administration of the dosage form to the patient.

3. The dosage form of claim 1, wherein the maximum plasma concentration of hydromorphone at the first Tmax (Cmax #1) is from about 1 to about 3 ng/ml, per administration of a 12 mg dosage of hydromorphone hydrochloride.

4. The dosage form of claim 1, wherein the second peak plasma concentration (Cmax #2) occurs in about 12.5 to about 16 hours after oral administration of the dosage form to the patient (Tmax #2).

5. The dosage form of claim 1, wherein the maximum plasma concentration of hydromorphone at Cmax #2 is from about 1.0 to about 3.6 ng/ml, per 12 mg hydromorphone hydrochloride administered over the 24 hour period.

6. The dosage form of claim 1, wherein the width of the plasma concentration curve at 50% of the height of the first Cmax (Cmax #1), based on a trough taken either as the Cmin between Cmax #1 and Cmax #2 or the plasma concentration at 24 hours after administration of the dose of hydromorphone) is from about 1.5 to about 4.5 hours.

7. The dosage form of claim 1, wherein the width of the plasma concentration curve at 50% of the height of the first Cmax (Cmax #1), based on a trough taken either as the Cmin between Cmax #1 and Cmax #2 or the plasma concentration at 24 hours after administration of the dose of hydromorphone) is from about 2.5 to about 3.5 hours.

8. The dosage form of claim 1, wherein the width of the plasma concentration curve at 50% of the height of the second Cmax (Cmax #2), based on a the trough taken either as the Cmin between Cmax #1 and Cmax #2 or the plasma concentration at 24 hours after administration of the dose of hydromorphone) is from about 4.5 to about 9 hours.

9. The dosage form of claim 1, wherein the width of the plasma concentration curve at 50% of the height of the second Cmax (Cmax #2), based on a the trough taken either as the Cmin between Cmax #1 and Cmax #2 or the plasma concentration at 24 hours after administration of the dose of hydromorphone) is from about 5.5 to about 7 hours.

10. The dosage form of claim 1, which provid es a maximum hydromorphone plasma concentration which is less than twice the plasma level of hydromorphone at about 24 hours after administration of the dosage form.

11. The dosage form of claim 1, which provides a maximum hydromorphone plasma concentration which is less than twice the plasma level of hydromorphone at the Cmin which occurs between Cmax #1 and Cmax #2.

12. The dosage form of claim 1, which provides an in-vitro dissolution of from about 5% to about 25% hydromorphone released after 1 hour; from about 40% to about 75% hydromorphone released after 8 hours; and not less than about 80% hydromorphone released after 18 hours.

13. The dosage form of claim 1, which provides an in-vitro dissolution of from about 10% to about 30% hydromorphone released after 2 hours; from about 40% to about 70% hydromorphone released after 8 hours; and at least about 80% hydromorphone released after 22 hours.

14. A solid sustained release once-a-day oral dosage form comprising hydromorphone or a pharmaceutically acceptable salt thereof together with a sustained release carrier, the dosage providing a relatively rapid rise in plasma concentration to a first initial early peak concentration (Cmax #1) in about 0.3 to about 4 hours after oral administration of the dosage form, followed by a second peak concentration (Cmax #2) which occurs in about 10 to about 19 hours after oral administration of the dosage form, said dosage form providing a maximum hydromorphone plasma concentration which is less than twice the plasma level of hydromorphone at about 24 hours after administration of the dosage form, said dosage form providing effective treatment of pain for about 24 hours or more after administration to a human patient.

15. The dosage form of claim 14, wherein the width of the plasma concentration curve at 50% of the height of the first Cmax (Cmax #1), based on a trough taken either as the Cmin between Cmax #1 and Cmax #2 or the plasma concentration at 24 hours after administration of the dose of hydromorphone) is from about 1.5 to about 4.5 hours.

16. The dosage form of claim 14, wherein the width of the plasma concentration curve at 50% of the height of the second Cmax (Cmax #2), based on a the trough taken either as the Cmin between Cmax #1 and Cmax #2 or the plasma concentration at 24 hours after administration of the dose of hydromorphone) is from about 4.5 to about 9 hours.

17. A solid sustained release once-a-day oral dosage form comprising hydromorphone or a pharmaceutically acceptable salt thereof together with a sustained release carrier, the dosage providing a first peak concentration (Cmax #1), followed by a second peak concentration (Cmax #2), said dosage form providing a maximum hydromorphone plasma concentration (i) which is less than twice the plasma level of hydromorphone at the Cmin which occurs between Cmax #1 and Cmax #2, and (ii) which is less than twice the plasma level of hydromorphone at about 24 hours after administration of the dosage form, said dosage form providing effective treatment of pain for about 24 hours or more after administration to a human patient.

18. The dosage form of claim 17, wherein the width of the plasma concentration curve at 50% of the height of the first Cmax (Cmax #1), based on a trough taken either as the Cmin between Cmax # 1 and Cmax #2 or the plasma concentration at 24 hours after administration of the dose of hydromorphone) is from about 1.5 to about 4.5 hours.

19. The dosage form of claim 18, wherein the width of the plasma concentration curve at 50% of the height of the second Cmax (Cmax #2), based on a the trough taken either as the Cmin between Cmax #1 and Cmax #2 or the plasma concentration at 24 hours after administration of the dose of hydromorphone) is from about 4.5 to about 9 hours.

20. The dosage form of claim 18, which provides Cmax #1 in about 0.3 to about 4 hours after oral administration of the dosage form, and which provides Cmax #2 in about 10 to about 19 hours after oral administration of the dosage form.
